(19) United States
(12) Patent Application Publication
(10) Pub. No.: US 2014/0121156 A1
(43) Pub. Date: May 1, 2014

(54) NUTRITIONAL APPROACH TO THE CONTROL OF ANEMIA, DIABETES AND OTHER DISEASES OR CONDITIONS AND PREVENTION OF ASSOCIATED COMORBID STATES WITH THE USE OF ERGOTHIONEINE

(71) Applicant: Marvin S. Hausman, Sherwood, OR (US)

(72) Inventor: Marvin S. Hausman, Sherwood, OR (US)

(73) Assignee: Entia Biosciences, Inc., Sherwood, OR (US)

(21) Appl. No.: 14/125,820

(22) PCT Filed: Jun. 13, 2012
(86) PCT No.: PCT/US12/42131

§ 371 Date: Dec. 12, 2013

Related U.S. Application Data
(60) Provisional application No. 61/496,321, filed on Jun. 13, 2011.

(51) Int. Cl. A61K 31/593; A61K 31/592; A61K 38/18; A61K 31/4164

(52) U.S. Cl. CPC A61K 31/593; A61K 31/4164; A61K 31/592; A61K 38/1816
USPC 514/5.4; 514/167; 514/6.9

(57) ABSTRACT

Nutritional products, compositions, pharmaceutical preparations and methods of use are disclosed for the prevention, suppression and treatment of anemia and/or diabetes and its various associated comorbidities. Uses of Ergothioneine to neutralize free radicals and/or cytokines, reduce oxidative stress, prevent inflammation, stimulate red blood cell production with increased levels of hemoglobin, and/or stabilize iron in its normal 2⁺ charge for proper oxygen binding and carrying are further disclosed. The extraction of Ergothioneine from whole food sources and bacterium for use in nutritional products, compositions, pharmaceutical preparations and treatments is also disclosed.

### FIELD OF THE INVENTION

This invention relates to whole foods, extracted ingredients, compositions, including nutritional products for preventing, suppressing, treating or controlling anemia and the various associated comorbidities of anemia, by the use of L-Ergothioneine (also referred to as Ergothioneine or ET), Vitamin D2 (Ergocalciferol) and/or other antioxidants to neutralize free radicals and/or cytokines, prevent inflammation, stimulate red blood cell production with increased levels of hemoglobin, and/or stabilize iron in its normal 2⁺ charge for proper oxygen binding and carrying. The extraction of Ergothioneine and Vitamin D2 from whole food sources and bacterium for use in nutritional products and treatments is also disclosed.

### BACKGROUND OF THE INVENTION

Early scientific studies relating to Ergothioneine identify its possible role as an antioxidant, through the incorporation of iron into the “heme” molecule (Goldberg, A. Brit. J. Haemat., 150-153 (1959)). It was suggested that Ergothioneine plays a part in the maintenance of hemoglobin iron in the reduced state. This potent antioxidant also appears to have a role in maintaining the function of erythrocytes and protecting them from oxidative damage (Touster, J. of Biol. Chem., 371 (1951); Chapman, P. K. Biomed. Biochem. Acta., 1143-1149 (1983)). The ability of Ergothioneine to protect hemoproteins such as hemoglobin within erythrocytes against oxidation probably could explain the millimolar concentrations seen in these cells (Arduini, A., Arch. Biochem. Biophys., 398-402 (1992); Spicer, S. W., Proc. Soc. Exp. Biol. & Med., 418-420 (1951)). The avidity by which dietary Ergothioneine is incorporated into tissues, the tenacity with which it is retained and its unique non-uniform pattern of tissue distribution support the physiological importance of this molecule.

A unique Ergothioneine Transporter (ETT) has been identified in human cells with the gene, SLC22A4 coding for an integral membrane protein, OCTN1, and the key substrate of this transporter is L-Ergothioneine (ET) (Grundemann, D. H., PNAS, 5256-5261 (2005)). The ETT is described in further detail in PCT/EP2005/005613 and U.S. patent application Ser. No. 11/569,451, titled “Identification of Ergothioneine Transporter and Therapeutic Uses Thereof,” such references are incorporated herein by reference in its entirety. ETT was identified as the first molecular marker of Ergothioneine activity proving to be necessary for the supply of ET primarily to erythrocyte progenitor cells and to monocytes. Using real-time PCR, strong expression of ETT in bone marrow was found (Kobayashi D, Ezp. Hematol., 1156-62 (2004)), suggesting that ETT charges developing erythrocytes with available ET, protecting erythrocytes against damage related to HbFeIV-O (ferryl hemoglobin). HbFeIV-O species is a highly reactive intermediate in the autocatalytic oxidation, caused by many xenobiotics, of HbFeIIO2 to methemoglobin (HbFeIII) and is also considered a starting point for detrimental radical reactions including heme degradation (Alayash, A., Nat. Rev., 152-159 (2004)).

Further data on the important role of Ergothioneine as a natural cytoprotectant is established. (Paul, B., Cell Death and Differentiation, 1-7 (2009)). Using RNA interference, cells were depleted of its transporter and cells lacking ETT were more susceptible to oxidative stress, resulting in mitochondrial DNA damage, protein oxidation and lipid peroxidation. ET was found to be as potent as glutathione, leading to the discovery that Ergothioneine may represent a new vitamin whose physiologic roles include antioxidant cytoprotection.

Ergothioneine is a unique, naturally occurring antioxidant that is found in most plants and animals, but highly concentrated in mushrooms. It has been established that Ergothioneine cannot be synthesized by humans and therefore is available only from dietary sources, which was confirmed in human bioavailability studies conducted in the Department of Food Science, Pennsylvania State University. (Weigand-Heller et al., Preventive Medicine, Vol. 54, Supplement 1:S75-S78 (2012)). A postprandial time course study of varying mushroom doses (0 g, 8 g, and 16 g) was used to evaluate the bioavailability of L-Ergothioneine (ET) from mushrooms in healthy men, using a randomized, cross-over, dose-response, postprandial time-course design. ET was administered through a mushroom test meal containing 8 g and 16 g of mushroom powder, equivalent to about 1 or 2 servings of fresh mushrooms respectively. Postprandial red blood cell concentrations of ET were measured. Plasma glucose, triglycerides, HDL, LDL and total cholesterol also were monitored. Biomarkers of inflammation and oxidative stress were evaluated using C-reactive protein and ORACₜₒₜₐₗ. According to the results, ET was bioavailable and a trend in the postprandial triglyceride response indicated that there was a blunting effect after both the 8 g and 16 g ET doses compared with the 0 g dose. Despite ET's antioxidant properties, ORACₜₒₜₐₗ values decreased after the 8 g and 16 g mushroom meal. The investigators stated that ET exerts antioxidant properties through multiple mechanisms aside from scavenging free radicals and that due to the various mechanisms of action, antioxidant capacity would be better measured by an oxidative stress biomarker.

This study convincingly indicated that L-Ergothioneine is bioavailable in humans through the consumption of mushrooms (peak of ET appeared in red blood cells (RBC) after only 2 hours of mushroom consumption), providing further supportive evidence for the ETT active transporter. The appearance of ET within red blood cells in such a short time after ingestion of mushrooms strongly suggests that human tissues and cells contain an active mechanism of transport for ET

These and other valuable health benefits of ET-enhanced mushrooms are disclosed in U.S. patent application Ser. Nos. 12/887,276 and 12/386,810, titled “Vitamin D2 Enriched Mushrooms and Fungi for Treatment of Oxidative Stress, Alzheimer's Disease and Associated Disease States,” and “Methods and Compositions for Improving the Nutritional Content of Mushrooms and Fungi,” respectively, which are herein incorporated by reference in its entirety. Mushrooms are a valuable health food—low in calories, high in vegetable proteins, chitin, iron, zinc, fiber, essential amino acids, vitamins and minerals. They are also an excellent source of organic selenium compounds, riboflavin, pantothenic acid, copper, niacin, potassium and phosphorous. Selenium is needed for the proper function of the antioxidant system, which works to reduce the levels of damaging free radicals in the body. Selenium is a necessary cofactor of one of the body's most important internally produced antioxidants, glutathione peroxidase, and also works with vitamin E in numerous vital antioxidant systems throughout the body. Mushrooms are also a primary source of natural Vitamin D, in the form of D2, which is naturally present in very few foods. Most other natural food sources of Vitamin D, in the form Vitamin D3, are of animal, poultry or seafood origin.

Vitamin D is a fat-soluble vitamin that is naturally present in very few foods, added to others, and available as a dietary supplement. Vitamin D comes in two forms (D2 (ergocalciferol) and D3 (cholecalciferol)) which differ chemically in their side chains. These structural differences alter their binding to the carrier protein Vitamin D binding protein (DBP) and their metabolism, but in general the biologic activity of their active metabolites is comparable. It is also produced endogenously when ultraviolet rays from sunlight strike the skin and trigger Vitamin D synthesis. So one must either ingest Vitamin D or sit in the sun and soak up UV rays, so that it may be synthesized endogenously. Most of the population is deficient in Vitamin D. The risks of sun exposure continue to gain attention, including the association of sun exposure with pre-cancerous (actinic keratosis) and cancerous (basal cell carcinoma, squamous cell carcinoma and melanoma) skin lesions—caused by loss of the skin's immune function, fine and coarse wrinkling of the skin, freckles, discoloration of the skin, and Elastosis (the destruction of the elastic tissue causing lines and wrinkles) is well documented. Thus, as people become more sensitive to the dangers of UV exposure, other dietary sources of Vitamin D become increasingly important for maintaining health.

There are two basic types of Vitamin D. Ergosterol is the basic building block of Vitamin D in plants and fungi. Cholesterol is the basic building block of Vitamin D in humans. When ultraviolet light from the sun hits the leaf of a plant or fungal tissue, ergosterol is converted into ergocalciferol, or Vitamin D2. In just the same way, when ultraviolet light hits the cells of our skin, one form of cholesterol found in our skin cells—called 7-dehydrocholesterol can be converted into cholecalciferol, a form of Vitamin D3. The liver and other tissues metabolize Vitamin D, whether from the skin or oral ingestion, to 25OHD, the principal circulating form of Vitamin D, by the enzyme CYP27B1, the 25OHD-1αhydroxylase. 25OHD is then further metabolized to 1,25(OH)2D principally in the kidney, although other tissues such as epidermal keratinocytes and macrophages contain this enzymatic activity. 1,25(OH)2D is the principal hormonal form of Vitamin D, responsible for most of its biologic actions.

Vitamin D has many roles in human health, including modulation of neuromuscular and immune function, reduction of inflammation, maintaining blood levels of phosphorus and calcium, promotion of bone mineralization and calcium absorption, maintaining a healthy immune system, and regulating cell differentiation and growth. Recent studies have also shown a link between vitamin D deficiency and diseases such as cancer, chronic heart disease, inflammatory bowel disease and even mental illness. In addition, many genes encoding proteins that regulate cell proliferation, differentiation, and apoptosis are modulated in part by Vitamin D. Many laboratory-cultured human cells have Vitamin D receptors and some convert 25(OH)D to 1,25(OH)₂D. It remains to be determined what cells, tissues, and organs in the human body contain either D2, D3, or both vitamin receptors and what additional cells with Vitamin D receptors in the intact human can carry out this conversion from 25(OH)D to 1,25(OH)₂D.

The detrimental effects of inflammatory conditions involve interactive processes involving inflammation, free radicals, reactive oxygen species (ROS) and oxidative stress. Free radicals (or ROS) are unstable, short lived and highly reactive and are biologic markers of various inflammatory conditions, including for example, cytokines such as IL-2, TNF-alpha, nitric oxide, hydrogen peroxide and heat shock protein. The effects of inflammatory processes and tissue damage caused by oxidative stress, free radicals and inflammatory processes relating to neuroinflammatory conditions are disclosed in U.S. patent application Ser. Nos. 12/887,276 and 13/363,579, titled “Anti-Inflammatory Approach to Prevention and Suppression of Post-Traumatic Stress Disorder, Traumatic Brain Injury, Depression and Associated Disease States,” which are herein incorporated by reference in their entirety.

There is additional evidence of the link between inflammatory processes and other disease states, such as diabetes. Researchers have concluded that “Obesity induces an insulin-resistant state in adipose tissue, liver, and muscle and is a strong risk factor for the development of type 2 diabetes mellitus. Insulin resistance in the setting of obesity results from a combination of altered functions of insulin target cells and the accumulation of macrophages that secrete proinflammatory mediators. Strategies focused on inhibiting the inflammation/insulin resistance axis that otherwise preserve essential innate immune functions may hold promise for therapeutic intervention.” (Olefsky, J. M. & Glass, C. K., Annu. Rev. Physiol., 2010; 72:219-46). As a result, the medicinal properties and usage of phytonutrients (or phytochemicals) in combination with Ergothioneine (L-Ergothioneine (ET)) and Vitamin D have utility for treating such inflammatory conditions and associated insulin resistance.

In addition, a recent Diabetes Prevention Program Outcomes Study (DPPOS) revealed that “diabetes risk during DPPOS was 56% lower for participants who had returned to normal glucose regulation versus those who consistently had pre-diabetes.” The research group concluded that pre-diabetes is a high-risk state for diabetes and that even transient reversion to normal glucose regulation is associated with a significantly reduced risk of future diabetes (independent of previous treatment). (Perreault et al., Diabetes Prevention Program Research Group, The Lancet, Jun. 9, 2012). As a result, the medicinal properties and usage of phytonutrients (or phytochemicals) in combination with Ergothioneine (L-Ergothioneine (ET)) and Vitamin D have utility assisting pre-diabetics to revert to normal glucose regulation and prevent progression to full-blown diabetes.

It is an object of the present invention to provide a natural, cost effective, natural whole food method to treat anemia and/or diabetes and prevent the comorbidities associated with anemia and/or diabetes.

It is a further object of the invention to provide a composition, such as Ergo-D2™, a potent anti-oxidant, anti-inflammatory nutritional product, to increase numbers and quality of red blood cells and mean corpuscular hemoglobin concentration (MCHC) and decrease total reliance on recombinant erythropoietin use in anemia patients.

It is a still further object of the invention to provide novel uses for anti-inflammatory effects of Ergothioneine for prevention, treatment and suppression of anemia and/or diabetes.

It is a still further object of the invention to provide to provide a composition, such as ErgoD2™ to inhibit the inflammation/insulin resistance axis in diabetes while at the same time preserving essential innate immune functions. A further object of the present invention is to provide a dietary supplement or other food or beverage products which are high in nutritional values, particularly Vitamin D2 and Ergothioneine that is extracted from natural whole food sources (including mushrooms, e.g. ErgoD2) and/or bacterial sources.

It is another object of the invention to provide dietary supplements, dietary ingredients or other food or beverage products obtained from whole, natural sources (such as Spirulina or oats) for use in prevention, suppression or treatment of anemia, diabetes and/or other inflammatory conditions.

It is an object of the present invention to diagnose the presence, absence, as well as varying concentrations of the Ergothioneine Transporter SLC22A4 within the membranes of cells and/or mitochondria in various human disease states, including for example autoimmune diseases (e.g. diabetes mellitus, rheumatoid arthritis, Crohn's disease), anemia (autoimmune or otherwise), kidney disease (autoimmune or otherwise) and other diseases.

These and other objects of the present invention will become apparent from the description of the invention which follows.

### SUMMARY OF THE INVENTION

Prevention, treatment and suppression of anemia and/or diabetes and the various comorbidity states are provided according to the invention. According to an embodiment, the invention creates an improved food or supplement product with a naturally enriched Vitamin D and Ergothioneine nutritional profile. According to an embodiment, the invention creates an improved food or supplement product with Ergothioneine, and optionally including Vitamin D and/or other antioxidants. The products according to the invention may be obtained from a variety of whole natural sources, including mushrooms, yeast, oats or barley or cyanobacteria, including Spirulina. The Ergothioneine may be combined with phytonutrients, Vitamin D enriched mushroom substrates (namely a mushroom or other fungi having enhanced content of Vitamin D or its analogs or derivatives), beta glucans and/or other antioxidants such as turmeric and/or n-acetyl cysteine.

In an embodiment, the combination of Ergothioneine and Vitamin D reduces the requirements for recombinant erythropoietin or other erythropoiesis-stimulating agents (ESA), providing a significant clinical benefit in the treatment of anemia. The compositions according to the invention may be provided as a daily supplementation regimen for prevention and/or as treatment regimens. In a further embodiment of the invention, the supplements or food product prevents, reduces and/or suppresses inflammation, oxidative stress and damage to blood cells, neutralizes pro-inflammatory signaling molecules, such as cytokines, and induces production of protective antioxidants, such as glutathione and IL6.

It is a still further embodiment, that the combination of Ergothioneine and Vitamin D, such as ErgoD2™, has the ability to inhibit the inflammation/insulin resistance axis in diabetes while at the same time preserving essential innate immune functions, resulting in better physiologic response to production of natural insulin.

In a further embodiment, the invention includes pharmaceutical compositions for prevention of, treatment for, and resistance to the effects of anemia and/or diabetes and other forms of inflammation and oxidative stress.

### DETAILED DESCRIPTION OF THE FIGURES

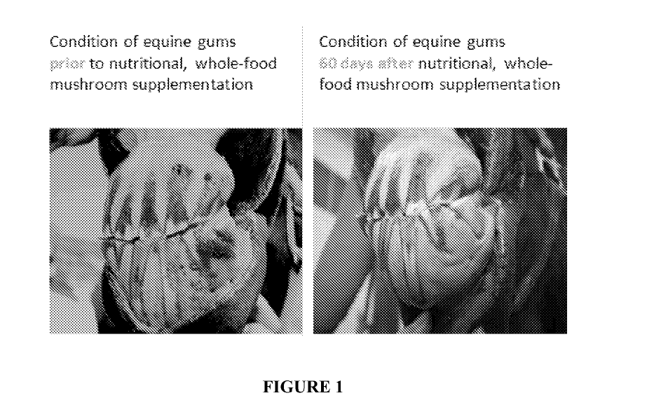

FIG. 1 shows the improvement in severity of gum disease in treated horses administered Ergothioneine according to an embodiment of the invention.

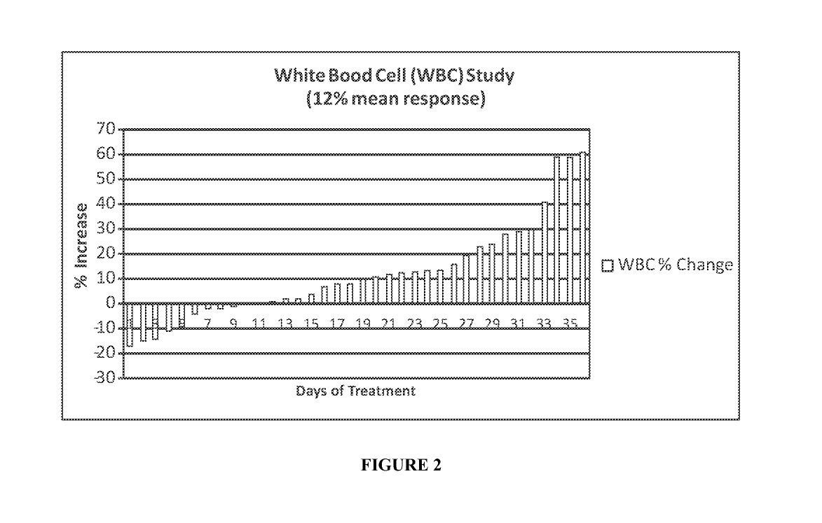

FIG. 2 shows results of the clinical marker of increased number of WBC in treated groups of horses.

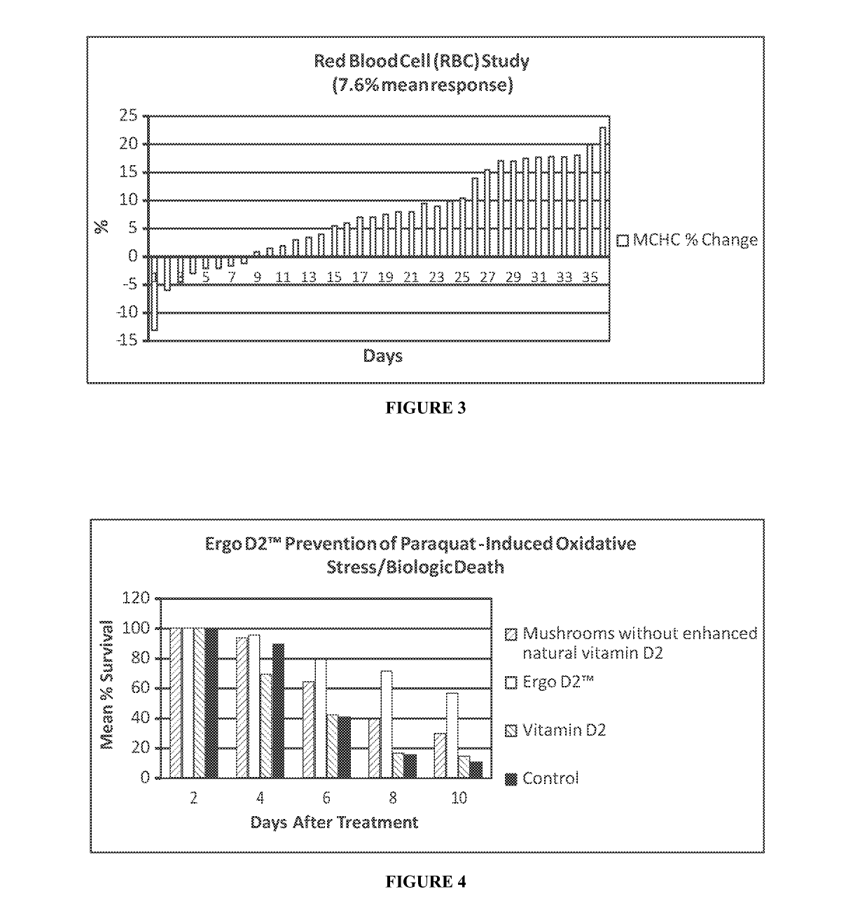

FIG. 3 shows the clinical marker of increased mean corpuscular hemoglobin concentration in treated groups of horses in an animal study according to an embodiment of the invention.

FIG. 4 shows the prevention of Paraquat-induced oxidative stress/biologic death by fungi with naturally-enriched Vitamin D2 based on mean percent survival.

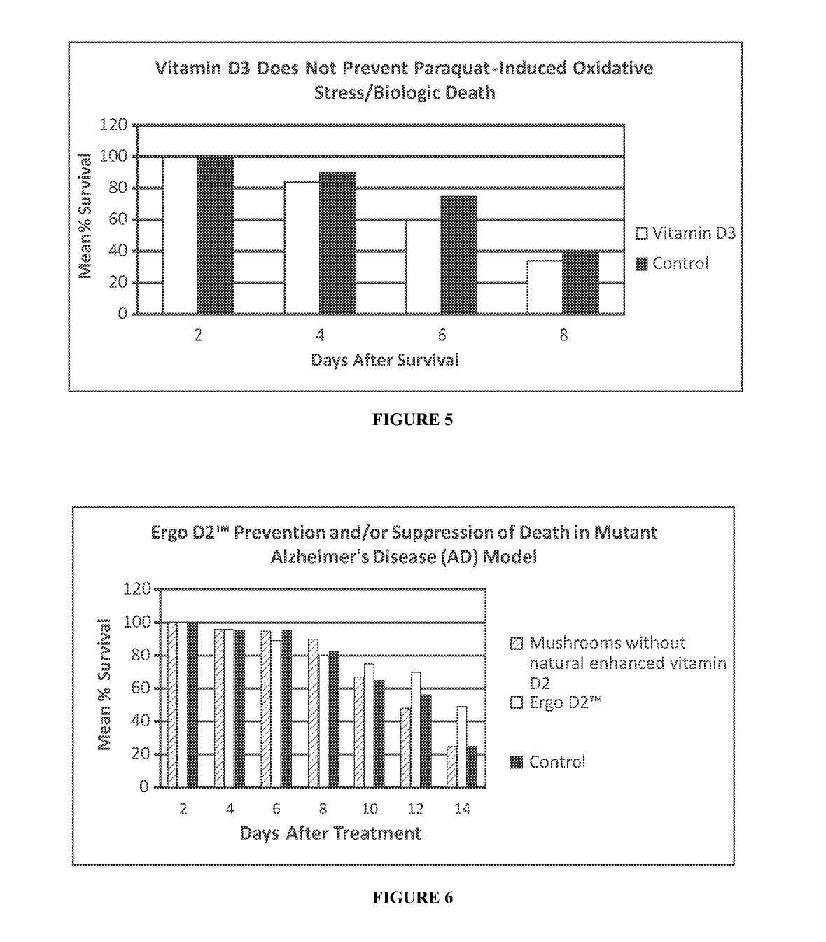

FIG. 5 shows the unexpected result that although Vitamin D2 within a whole food is able to counteract and/or neutralize the oxidative stress effect and resulted in a 30% increase in survival, pure Vitamin D2 and Vitamin D3 by itself have no effect on survival.

FIG. 6 shows the improvement in survival of mutant Alzheimer's Disease (AD) flies given A. blazei enriched with Vitamin D2, having a survival rate nearly double that of the control or A. blazei without any enrichment.

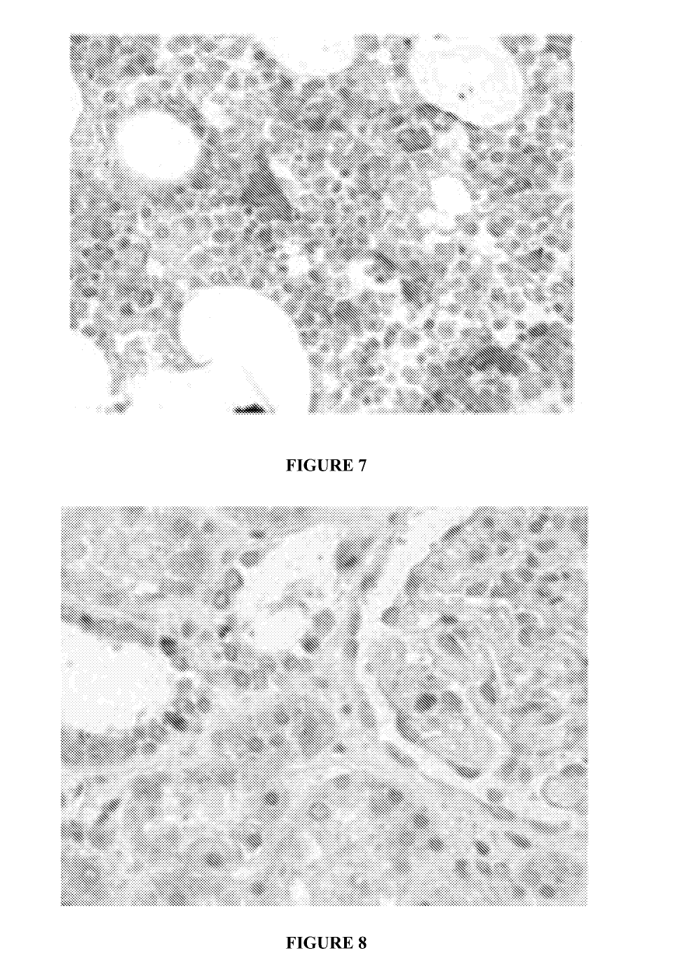

FIG. 7 shows immunohistochemistry study slides indicating the presence of ETT in normal bone marrow according to an embodiment of the invention.

FIG. 8 shows immunohistochemistry study slides indicating the presence of ETT in normal kidney according to an embodiment of the invention.

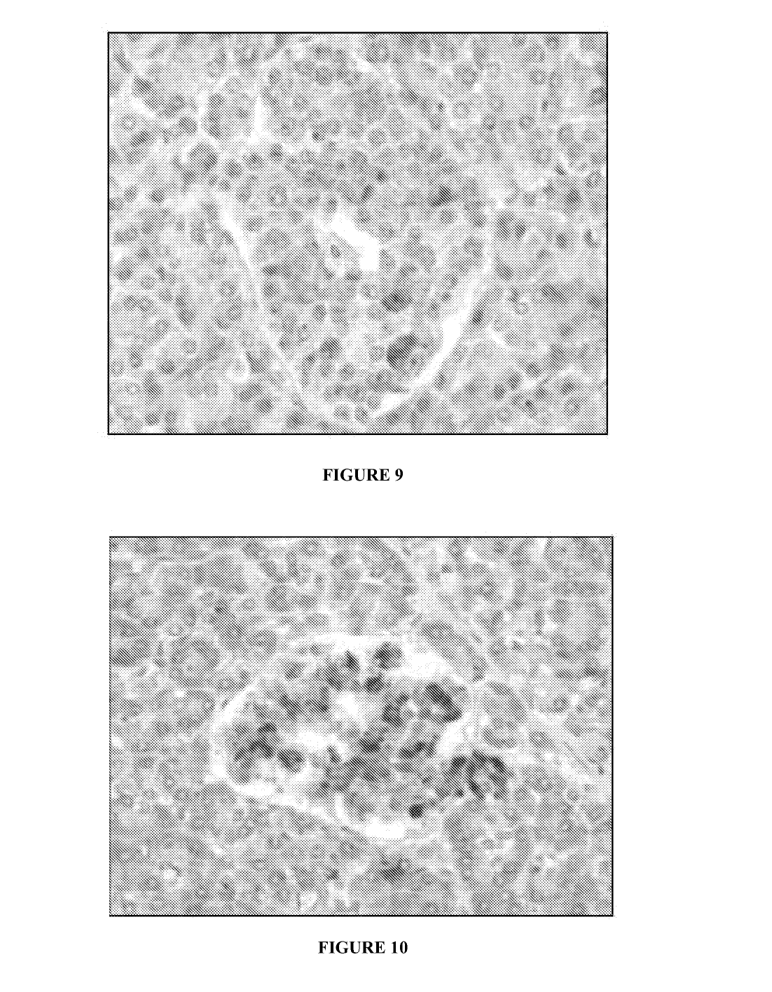

FIG. 9 shows immunohistochemistry study slides indicating the presence of ETT in normal pancreas according to an embodiment of the invention.

FIG. 10 shows immunohistochemistry study slides indicating the increased presence of ETT in a pancreas of a diabetic patient according to an embodiment of the invention.

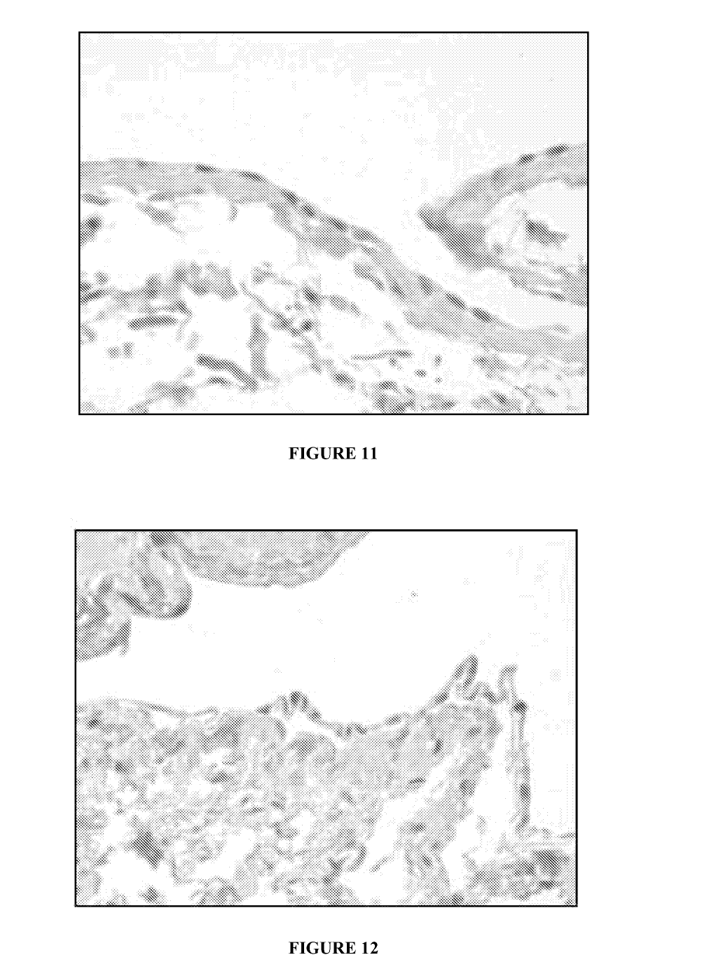

FIGS. 11-12 show immunohistochemistry study slides indicating the lack of staining of ETT in normal joint tissue according to an embodiment of the invention.

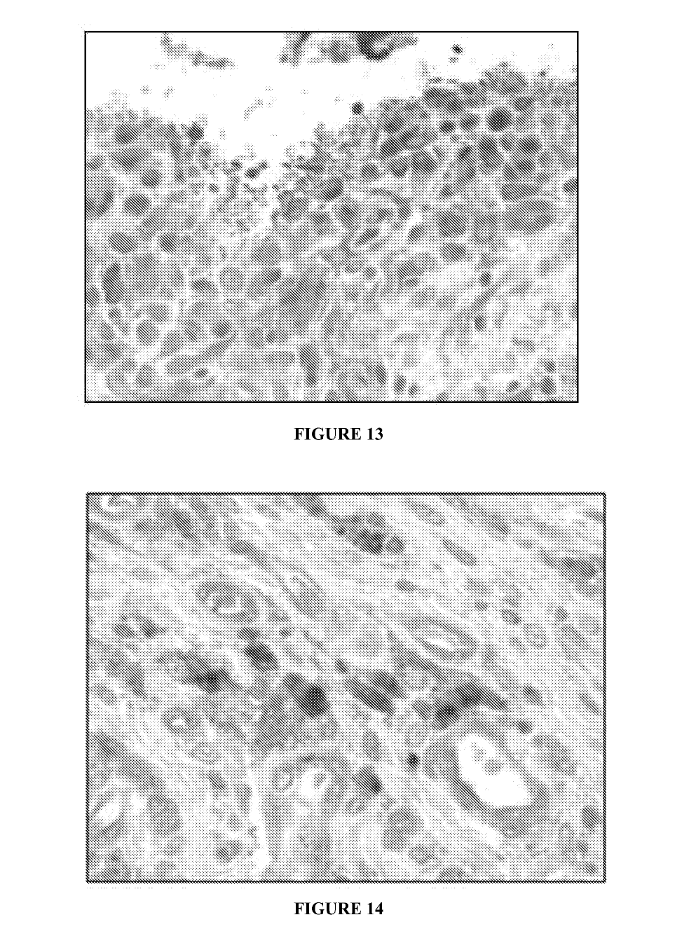

FIGS. 13-14 show immunohistochemistry study slides with heavily stained cells showing the presence of ETT in joint tissue of a patient having rheumatoid arthritis.

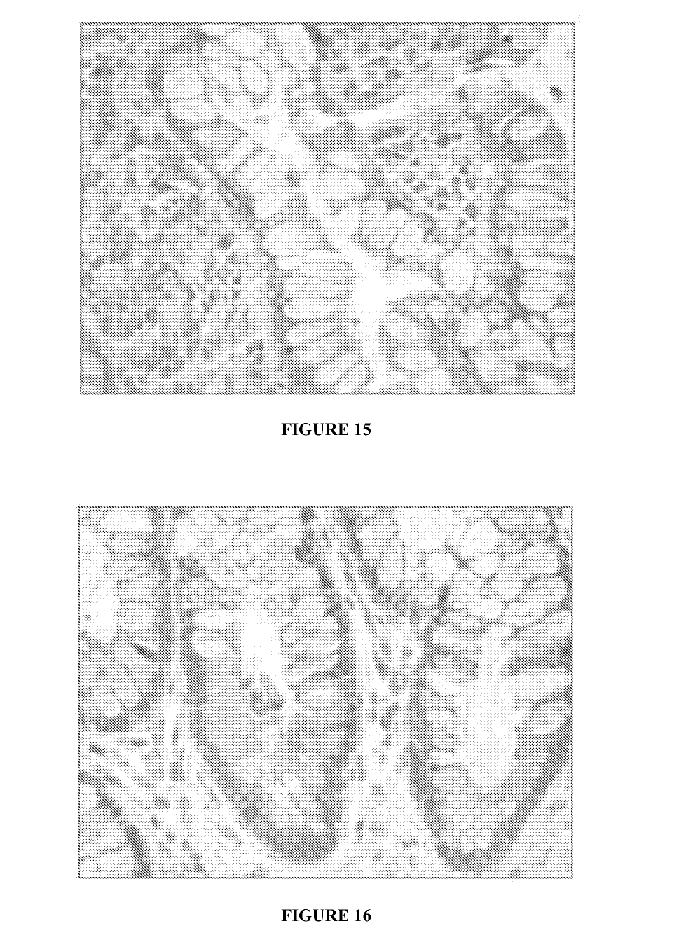

FIGS. 15-16 show immunohistochemistry study slides indicating the lack of staining of ETT in normal intestinal tissue according to an embodiment of the invention.

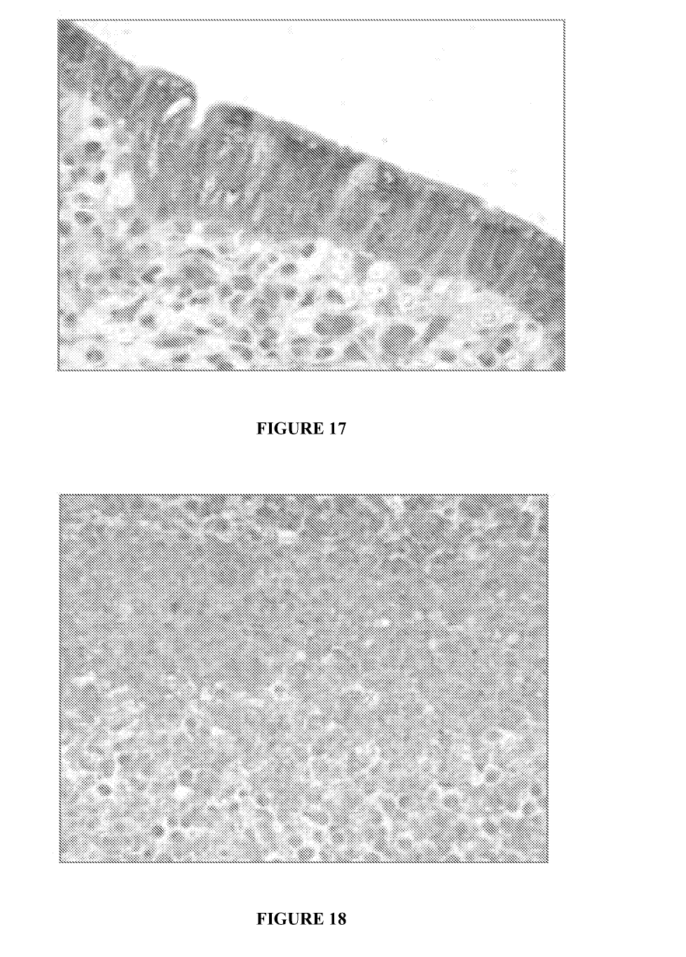

FIGS. 17-18 show immunohistochemistry study slides with heavily stained cells showing the presence of ETT in intestinal tissue of a patient having Crohn's disease.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION OF THE INVENTION

The embodiments of this invention are not limited to particular embodiments for compositions and uses of Ergothioneine for anemia and related comorbidities, which can vary and are understood by skilled artisans. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers defining the range and include each integer within the defined range.

Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about”, the claims include equivalents to the quantities refers to variation in the numerical quantity that can occur.

As used herein the term “anemia” refers to a decrease in number of red blood cells (erythrocytes) or less than the normal quantity of hemoglobin in the blood. Any abnormality in hemoglobin or erythrocytes results in reduced oxygen levels in the blood. Anemia can also include decreased oxygen-binding capacity of hemoglobin molecules due to deformity or abnormalities of hemoglobin binding of oxygen due to hemoglobin iron in the 3⁺ state. The iron atom in the heme group must initially be in the ferrous (Fe2+) oxidation state to support oxygen and other gases' binding and transport. Initial oxidation to the ferric (Fe3+) state without oxygen converts hemoglobin into “hemiglobin” or methemoglobin, which cannot bind oxygen. Hemoglobin in normal red blood cells is protected by a reduction system to keep this from happening. Anemia can also be associated with abnormal production, processing, or performance of erythrocytes and/or hemoglobin. The term anemia refers to any reduction in the number of red blood cells and/or level of hemoglobin in blood relative to normal blood levels. The term anemia as used also refers to the size of red blood cells and size is reflected in the term mean corpuscular volume (MCV). The classifications of anemia using MCV include macrocytic, normocytic and microcytic anemia. Kinetic approaches to defining anemia include analysis of the reticulocyte count which is a quantitative measure of the bone marrow's production of new red blood cells. The degree of anemia is assessed by measuring the reticulocyte production index which is a calculation of the ratio between the level of anemia and the extent to which the reticulocyte count has risen in response.

As one skilled in the art will appreciate, anemia can arise due to a variety of conditions such as acute or chronic kidney disease, infections, inflammation, cancer, irradiation, toxins, diabetes, and surgery. For example, infections may be due to, e.g. virus, bacteria, and/or parasites, etc. Inflammation may be due to acute or chronic trauma, infection, autoimmune disorders, such as rheumatoid arthritis, autoimmune hemolytic anemia, transfusion reactions, etc. Anemia can also be associated with blood loss due to, e.g. stomach ulcer, duodenal ulcer, hemorrhoids, cancer of the stomach or large intestine, trauma, injury, surgical procedures, etc. Anemia is further associated with radiation therapy, chemotherapy, and kidney dialysis, e.g., chemotherapy-induced anemia, anemia associated with chronic kidney disease (CKD), HIV-infected patients undergoing treatment with azidothymidine (zidovudine) or other reverse transcriptase inhibitors, and can develop in cancer patients undergoing chemotherapy, e.g. with cyclic cisplatin- or non-cisplatin-containing chemotherapeutics. Aplastic anemia and myelodysplastic syndromes are diseases associated with bone marrow failure that result in decreased production of erythrocytes. Still further, anemia can result from defective or abnormal hemoglobin or erythrocytes, such as in disorders including microcytic anemia, hypochromic anemia, etc. Anemia can result from iron deficiency, either nutritionally based or related to disorders in iron uptake, mobilization, transport, processing, and utilization, see, e.g. sideroblastic anemia, etc. One skilled in the art shall appreciate the numerous applications for the compositions and the methods of use disclosed according to the present invention.

The term “anemia” is also understood to include anemic “conditions” and “disorders.” These further include any condition, disease, or disorder associated with anemia; including for example, aplastic anemia, autoimmune hemolytic anemia, bone marrow transplantation, Churg-Strauss syndrome, Diamond Blackfan anemia, Fanconi's anemia, Felty syndrome, graft versus host disease, hematopoietic stem cell transplantation, hemolytic uremic syndrome, myelodysplastic syndrome, nocturnal paroxysmal hemoglobinuria, osteomyelofibrosis, pancytopenia, pure red-cell aplasia, purpura Schoenlein-Henoch, sideroblastic anemia, refractory anemia with excess of blasts, rheumatoid arthritis, Shwachman syndrome, sickle cell disease, thalassemia major, thalassemia minor, thrombocytopenic purpura, etc.

As used herein the term “mushroom” or “filamentous fungi” shall be interpreted to include all tissues, cells, organs of the same, including but not limited to mycelium, spores, gills, fruiting body, stipe, pileus, lamellae, basidiospores, basidia, and the like.

As used herein the term “naturally-enhanced” with respect to whole foods such as mushrooms, yeast, cyanobacteria, Spirulina and Vitamin D, shall include pulsed UV irradiated mushrooms, yeast, cyanobacteria, Spirulina, etc. produced by the methods disclosed herein. The naturally-enhanced products according to the invention may include the enhanced whole food as well as powders and other forms obtained from the whole food.

The terms “subject” or “patient” are used herein interchangeably and as used herein mean any mammal including but not limited to human beings including a human patient or subject to which the compositions of the invention can be administered. The term “mammals” include human patients and non-human primates, as well as experimental animals such as rabbits, rats, and mice, and other animals.

The term “treating” or “treatment” as used herein, refers to any indicia of success in the prevention or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluations. Accordingly, the term “treating” or “treatment” includes the administration of the compounds or agents of the present invention which may be in combination with other compounds.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

Compositions

According to an embodiment of the invention, a nutritional supplement, ingredient, food or beverage composition and/or pharmaceutical composition for treating anemia and preventing the comorbid disease states associated therewith may include Ergothioneine, Vitamin D2 and/or D3, phytonutrients, beta glucans, omega-3 or alternative antioxidants, a pharmaceutically-acceptable carrier and/or combinations of the same.

As used herein the term Ergothioneine shall be interpreted to include variants, homologs, optical isomers and the like which retain the antioxidant activity of Ergothioneine or L-Ergothioneine as demonstrated and described herein. Ergothioneine is a naturally-occurring amino acid. Ergothioneine is a natural antioxidant but is unable to be made in human cells, rather it is absorbed from the diet. Ergothioneine from any suitable source may be used according to the invention. L-Ergothioneine is available commercially from Oxis International, Inc., Sigma Chemical, etc. or from dietary sources such as mushrooms and the various sources disclosed herein according to the invention. The compound is also available from Actinobacteria, filamentous fungi, cyanobacteria, Spirulina, oats, barley and other whole food sources. Ergothioneine for use in compositions according to the invention may be obtained from an independent bionutrient source, such as Vitamin D enriched mushrooms disclosed herein, whole food sources, cyanobacteria and Spirulina as disclosed according to the embodiments of the invention.

According to one embodiment of the invention, Vitamin D2 and/or D3 may be provided from a UV irradiated, Agaricus fungi, tissue, substrate or component thereof with higher levels of Vitamin D2 than a non-irradiated product. According to an embodiment of the invention, the novel mushroom whole food (Ergo-D2™) may be used. Ergo-D2™ contains high levels of three bioactive components previously shown to have health promoting properties—-Vitamin D2, L-Ergothioneine (ET) and beta-glucans.

Vitamin D and Ergothioneine enriched mushrooms according to the invention are pulsed with UV light at lower ranges and for very brief periods have increases by as much as 800 times the % DV (percent daily value) of Vitamin D content, per serving with no deleterious effects on the morphology or appearance of the mushroom. Pulsed UV-light treatments to increase Vitamin D₂ content in mushrooms were conducted with a laboratory scale, pulsed light sterilization system (SteriPulse®-XL 3000, Xenon Corporation, Woburn, Mass.) that is present in the Department of Agricultural Biological Engineering at Penn State. While applicants postulate that it is the UVB component of the Xenon pulsed light system that is responsible for the effects of the invention, it should be noted that the system uses pulsed light which includes the entire spectrum of light and may also include other components that contribute to the effects demonstrated herein and which are intended to be within the scope of the invention.

Any type of mushroom, mushroom part, component, fungi or even used substrate for cultivating mushrooms, with ergosterol present may be used. This includes all filamentous fungi where ergosterol has been shown to be present and includes the use of tissues such as the mycelia, spores or vegetative cells. This includes, but is not limited to, for example, Coprinus, Agrocybe, Hypholoma, Hypsizygus, Pholiota, Pleurotus, Stropharia, Ganoderma, Grifola, Trametes, Hericium, Tramella, Psilocybe, Agaricus, including for example Agaricus bisporus (e.g. white button mushrooms), Phytophthora achlya, Flammulina, Melanoleuca, Agrocybe, Morchella, Mastigomycotina, Auricularia, Gymnopilus, Mycena, Boletus, Gyromitra, Pholiota, Calvatia, Kuegneromyces, Phylacteria, Cantharellus, Lactarius, Pleurotus, Clitocybe, Lentinula (Lentinus), Stropharia, Coprinus, Lepiota, Tuber, Tremella, Drosophia, Leucocoprinus, Tricholoma, Dryphila, Marasmius, and Volvariella.

In addition, the solid substrate can be any part of the mushroom or mold, including the mycelia, spores etc., so long as ergosterol is present in at least part of the tissue or cells. In yet another embodiment, the spent mushroom substrate upon which mushrooms are cultivated, was enriched in Vitamin D using pulsed UV light according to the invention. As one skilled in the art shall ascertain, mushrooms are usually produced by first preparing a substrate, such as corn, oats, rice, millet or rye or various combinations, prepared by soaking the grain in water and sterilizing the substrate before inoculation with mushroom spores or mushroom mycelia. Mycelia are the filamentous hyphae of a mushroom that collect water and nutrients to enable mushrooms to grow. The inoculated substrate is then held to promote colonization of the mycelia, at which point the mycelia-laced grains become “spawn”. This is usually done in individual spawn bags. The substrate provides the nutrients necessary for mycelium growth. The mycelium-impregnated substrate then develops under controlled temperature and moisture conditions, until the hyphae of the mycelium have colonized the substrate. The mycelium enriched product usually is harvested after about four to eight weeks from the beginning of the process, with the contents of the spawn bag possibly processed into dry powdered product. According to the invention, this spent substrate may also be enriched in Vitamin D upon application of pulsed UV irradiation.

Non-limiting examples of other fungal genera, including fermentable fungi, include: Alternaria, Endothia, Neurospora, Aspergillus, Fusarium, Penicillium, Blakeslea, Monascus, Rhizopus, Cephalosporium, Mucor, and Trichoderma.

In addition to the irradiated mushrooms according to an embodiment of the invention for providing a composition with enhanced Ergothioneine and Vitamin D, additional substrates for Ergothioneine may be irradiated to enhance the Ergothioneine content, including for example cyanobacteria and Spirulina. According to a further embodiment of the invention, cyanobacteria and/or Spirulina may be added as an additive ingredient to the irradiated mushrooms. According to a further embodiment of the invention, cyanobacteria and/or Spirulina may be irradiated and added to irradiated mushrooms.

Additional antioxidants may be beneficial in the compositions according to the invention. For example, turmeric and its active component curcumin are phytonutrients that act as antioxidants. According to an embodiment, the compositions of the invention comprise a phytonutrient antioxidant in addition to the fungi component to provide a combined synergistic response.

An example of a suitable phytonutrient according to the invention is turmeric. Tumeric is available in various forms contains up to 5% essential oils and up to 5% curcumin, a polyphenol. Curcumin is the active substance of turmeric and curcumin is known as C.I. 75300, or Natural Yellow 3. The systematic chemical name is (1E,6E)-1,7-bis(4-hydroxy-3-methoxyphenyl)-1,6-heptadiene-3,5-dione and exists in tautomeric forms—keto and enol.

Food or Beverage Compositions

An embodiment of the present invention also provides natural bionutrients, medical foods and/or beverages comprising combinations of Ergothioneine, enriched mushrooms of the invention including extracts, fractions thereof or compounds thereof or any combination thereof, phytonutrients and/or antioxidants. The food compositions according to the invention may comprise enriched mushrooms from a variety of fungi sources as disclosed according to embodiments herein this description. Alternatively, the food compositions according to the invention may comprise Ergothioneine obtained directly from whole food sources, Spirulina or cyanobacteria.

The medical food is compounded for the amelioration of a disease, disorder or condition associated with or caused by inflammation, oxidative stress and/or decreased levels of Ergothioneine. According to a preferred embodiment of the invention, food compositions are intended for human consumption for daily supplementation. Ranges of the amounts of each component of the food compositions can be adjusted as necessary for the supplementation of individual patients and according to the specific condition treated. Any variations in the amount of the ingredients may be utilized according to the desired composition formulation.

The medical foods according to the invention are formulated to manage a specific disease or condition for which medical evaluation, based on recognized scientific principles, has established distinct nutritional requirements. All components of the medical foods have GRAS status (Generally Recognized as Safe) as designated by the FDA or independent review. In a preferred embodiment, a medical food according to the invention, ErgoD2™ Hemo, is an encapsulated medical food that is certified organic and may be dispensed by a medical practitioner as indicated for the distinct nutritional requirements of patients being treated for diabetes and/or anemia, as disclosed herein according to the methods of the invention.

The food composition according to the invention may be prepared by any of the well-known techniques known by those skilled in the art, consisting essentially of admixing the components, optionally including one or more accessory ingredients. In one embodiment, the extracts, fractions, and compounds of this invention may be administered in conjunction with other additives and fillers known to those of skill in the art. Other compatible actives may be included in the food compositions of the present invention.

According to one embodiment of the invention, a beverage composition is provided. For particularly suitable applications for patients suffering from anemia, such as dialysis patients, a beverage composition is provided on a daily basis. According to a further embodiment, a food supplement is provided on a daily basis, to ensure that the supplementation provides a whole food source of the Ergothioneine and Vitamin D. Although not intended to be limited according to a particular theory of the present invention, providing a whole food source administers various co-enzyme factors from the whole food providing additional supplementation and treatment benefits. According to an alternative embodiment, an extracted source of the Ergothioneine and Vitamin D (e.g. dried mushroom powder or Spirulina) can be added to the food or beverage composition.

Pharmaceutical Compositions

In an embodiment of the invention, a pharmaceutical composition for treating a disease state including anemia or diabetes. In an additional embodiment, a pharmaceutical composition for treating a disease state associated with inflammation, oxidative stress and/or decreased levels of Ergothioneine comprises a combination of the following ingredients (in a variety of combinations, such that not every component is required according to various embodiments of the invention), a source of Ergothioneine, a UV irradiated, enriched mushroom, tissue, substrate or component thereof with higher levels of Vitamin D2 than a non-irradiated product, and a pharmaceutically-acceptable carrier. The pharmaceutical compositions according to the invention may further comprise antioxidants, phytonutrients and other beneficial components for treatment of the conditions disclosed herein.

According to a further embodiment of the invention, the pharmaceutical composition may further comprise another bioactive nutrient attached to Ergothioneine. Although not intended to be limited to a particular theory of the invention, the attachment of a bionutrient to Ergothioneine delivers the bionutrient along with the Ergothioneine, wherein the Ergothioneine acts as an active carrier to deliver the bionutrient to a cell. According to an additional non-limiting theory of the invention, the ETT permits the bionutrient to enter the cell. For example, selenium and/or extracted products from beer hops, oats, barley, etc. can be added to the Ergothioneine and the pharmaceutical compositions of the invention.

The pharmaceutically-acceptable carrier according to the invention facilitates administration of the composition to a patient in need thereof. The turmeric, Ergothioneine and the compound, extracts, fractions and/or compounds derived therefrom the enriched mushrooms of the invention may be mixed with any of a variety of pharmaceutically-acceptable carriers for administration. “Pharmaceutically acceptable” as used herein means that the extract, fraction thereof, or compound thereof or composition is suitable for administration to a subject to achieve the treatments described herein, without unduly deleterious side effects in light of the severity of the disease and necessity of the treatment. According to the invention, the carrier may be a solid or a liquid, or both, and is preferably formulated with the compound as a unit-dose formulation, for example, a tablet, which may contain from 0.5% to 95% by weight of the active compound.

The pharmaceutical composition according to the invention may be prepared by any of the well-known techniques of pharmacy consisting essentially of admixing the components, optionally including one or more accessory ingredients. In one embodiment, the extracts, fractions, and compounds of this invention may be administered in conjunction with other medicaments known to those of skill in the art. Other compatible pharmaceutical additives and actives may be included in the pharmaceutically acceptable carrier for use in the compositions of the present invention.

Dose ranges of the pharmaceutical compositions can be adjusted as necessary for the treatment of individual patients and according to the specific condition treated. Any of a number of suitable pharmaceutical formulations may be utilized as a vehicle for the administration of the compositions of the present invention and maybe a variety of administration routes are available. The particular mode selected will depend of course, upon the particular formulation selected, the severity of the disease, disorder, or condition being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, nasal, transdermal or parenteral routes and the like. Accordingly, the formulations of the invention include those suitable for oral, rectal, topical, buccal, sublingual, parenteral (e.g., subcutaneous, intramuscular, intradermal, inhalational or intravenous) and transdermal administration, although the most suitable route in any given case will depend on the nature and severity of the condition being treated and on the nature of the particular active product used.

Formulations suitable for oral administration may be presented in discrete units, such as capsules, cachets, lozenges, drops, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above).

In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, inhalational or intradermal injection. Such preparations may conveniently be prepared by admixing the compound with water or a glycine buffer and rendering the resulting solution sterile and isotonic with the blood. Alternately, the extracts, fractions thereof or compounds thereof can be added to a parenteral lipid solution.

Formulations of the inventive mixtures are particularly suitable for topical application to the skin and preferably take the form of an ointment, cream, lotion, paste, gel, spray, aerosol, or oil. Carriers which may be used include Vaseline, lanoline, polyethylene glycols, alcohols, transdermal enhancers, and combinations of two or more thereof.

Formulations suitable for transdermal administration may also be presented as medicated bandages or discrete patches adapted to remain in intimate contact with the epidermis of the recipient for a prolonged period of time. Formulations suitable for transdermal administration may also be delivered by iontophoresis (passage of a small electric current to “inject” electrically charged ions into the skin) through the skin. For this, the dosage form typically takes the form of an optionally buffered aqueous solution of the active compound. Suitable formulations comprise citrate or bis/tris buffer (pH 6) or ethanol/water and contain from 0.01 to 0.2M active ingredient.

The therapeutically effective dosage of any specific compound will vary somewhat from compound to compound, patient to patient, and will depend upon the condition of the patient and the route of delivery. As a general proposition, a dosage from about 0.01 to about 50 mg/kg will have therapeutic efficacy, with still higher dosages potentially being employed for oral and/or aerosol administration. Toxicity concerns at the higher level may restrict intravenous dosages to a lower level such as up to about 10 mg/kg, all weights being calculated based upon the weight or volume of the enriched mushrooms, fractions thereof or compounds thereof of the present invention, including the cases where a salt is employed. In an aspect of the invention a pharmaceutical composition provided in 500 mg capsules may be dosed to a patient from 1 to 4 capsules a day, preferably 2 to 4 capsules a day.

In an aspect of the invention, the pharmaceutical composition provides a blend of mushroom antioxidants and optionally phytonutrients. In certain aspects, the pharmaceutical composition may be classified also as a medical food. High concentrations of natural Ergothioneine and Ergocalciferol (vitamin D2) are included in the compositions for administration to a patient in need thereof. In an aspect of the invention, the compositions may be formulated as vegan products. In an additional aspect of the invention, the compositions contain USDA certified organic ingredients and do not include any artificial colors, flavors, or preservatives. In a further aspect of the invention, the compositions provide a natural, non-toxic product.

Extraction of Ergothioneine from Various Sources for Use in Compositions

The isolation, extraction and/or sourcing of Ergothioneine from additional sources is disclosed according to the methods of use of the present invention. As a result, various whole sources of food and/or bacteria may be used to provide the Ergothioneine required for the methods of use and/or the compositions according to the invention.

Previously the extraction of Ergothioneine was achieved from the enriched mushroom sources disclosed herein. The mushrooms were further enriched with Vitamin D2 and/or D3 and could be obtained, for example, from a UV irradiated, Agaricus fungi, tissue, substrate or component thereof with higher levels of Vitamin D2 than a non-irradiated product. A preferred source for the enriched mushroom is the whole food (Ergo-D2™), containing high levels of three bioactive components—Vitamin D2, L-Ergothioneine (ET) and beta-glucans.

According to a further embodiment of the invention, Ergothioneine can further be obtained from cyanobacteria. Cyanobacteria can be used for extraction of Ergothioneine and/or a source for Ergothioneine. Spirulina is a blue-green algae that has been identified to be a source of Ergothioneine. Spirulina is a microscopic blue-green alga in the shape of a spiral coil, living both in sea and fresh water. It is the most common name for human and animal food or nutritional supplement made primarily from two species of cyanobacteria: Arthrospira platensis and Arthrospira maxima.

According to a further embodiment, various plant materials are used to source Ergothioneine for the methods of use and/or the compositions according to the invention. Plant material sources for Ergothioneine may include cereal grains, including oats, wheat and barley. Ergothioneine may be further extracted from beer hops, and cereal grains, including oats, barley, etc.

Upon extraction or isolation of Ergothioneine from a source additional molecules and entities can be attached to permit delivery into the cell along with the Ergothioneine. As is recognized in the art relating to Ergothioneine, ETT provides a mechanism of delivery of Ergothioneine within cells. As a result, it is desirable to attach additional molecules to Ergothioneine, upon isolation from at least the sources disclosed herein (e.g. whole foods and cyanobacteria), including for example, beta-glucans, antioxidants, selenium, phytonutrients, and/or vitamins, such as Vitamin C and Vitamin D2. The attachment of additional molecules to an extracted source of Ergothioneine permits the effective delivery into the mitochondria of the cells of a patient in need of treatment according to the embodiments of the invention.

Enhancement and Extraction of Vitamin D2 and Other Bioactive Ergosterol-Derived Products Following Pulsed UV Light Exposure of Mushrooms from Various Sources for Use in Compositions of the Invention.

Exposure of mushrooms to UV light irradiation generates, in addition to Vitamin D2, additional ergosterol derived products, such as pre-vitamin D2, lumisterol2 and tachysterol. Vitamin D2 is the most abundant product, followed by pre-vitamin D2, lumisterol2 and tachysterol2 (order of decreasing abundance). In addition, untreated mushroom samples did not contain detectable levels of any photoproduct. (Kalaras et al., Food Chemistry, (May 2012) In Press). This reference is herein incorporated by reference in its entirety.

As an embodiment of this invention, the use of UV enhanced mushrooms, including ErgoD2™ medical food, and/or extracts and the resultant physiologic effects may be associated not only with Vitamin D2 but also with ergosterol derived photoproducts.

Methods of Use—Anemia

Embodiments of the invention include methods of treating anemia, methods of decreasing inflammation and increasing resistance to oxidative stress and associated disease states. The methods of use disclosed herein may be used for treating all types of anemia, whether or not dialysis treatments are required, including for example, conditions such as anemia associated with kidney disease (e.g. chronic kidney diseases, stages 2-5 end-stage renal disease, etc.), anemia of chronic disease, anemia of cancer, chemotherapy-induced anemia, iron deficiency anemia and the like.

Often anemic patients are treated with the administration of erythropoiesis stimulating proteins (ESPs), including recombinant human erythropoietin (EPO) or Aranesp® (available from Amgen). The present invention improves upon prior research using Vitamin D with erythropoietin (EPO) for anemia patients. Several studies have shown that most hemodialysis patients are deficient in 25-hydroxyvitamin D (25-D) (Del Valle, Hemodial. Int., 315-321 (2007)). A recent safety and efficacy study of Vitamin D2 (ergocalciferol) in hemodialysis patients found a significant reduction in use of recombinant human erythropoietin (EPO) in treated patients (Saab G, Nephron Clin. Pract., 132-138 (2007)). In the study 64% of the patients had a reduction in EPO dose after D2 supplementation. EPO administration can assist in anemia management in hemodialysis patients but use of EPO also has complications; higher doses are an independent predictor of mortality (Zhang Y, Am. J. Kidney Dis., 866-876 (2004)). Another recent pilot study on 81 vitamin D-deficient dialysis patients receiving supplementation with Vitamin D2 evaluated EPO doses (Kumar, V. D., J. Nephrology, 98-105 (2011)). Although more than half the patients (57%) required less EPO with the supplementation of Vitamin D2, only 44% of the patients were able to reach 25-D levels of 30 ng/mL or greater.

The methods of treating anemia according to the invention improve upon these studies by providing Ergothioneine and/or the various compositions according to the invention. For example, the findings of Kumar, V. D. are improved upon by combining ergothioneine with Vitamin D2 replacement, which addresses the inflammatory reactions taking place in the bone marrow with resultant inhibition of red blood cell production. As nearly all dialysis patients are susceptible to inflammatory reactions in their blood vessels, poor immunity, increased formation and release of toxic cytokines (free radicals), etc., the addition of ET to Vitamin D is useful in further neutralizing toxic free radicals associated with inflammation.

Without being limited to a particular theory of the invention, the use of the compositions for treatment of anemia supplies electrons to stabilize and reactivate hemoglobin, in addition to maintaining iron in the +2 oxidation state required for oxygen binding. The treatments according to the invention may also stimulate progenitor bone marrow stem cells to increase production of red cells. These and other benefits of using Ergothioneine and/or the various compositions according to the invention indicate the usefulness in treating anemia.

In a further aspect of the invention, the methods of treating anemia demonstrate improved blood cell counts and hemoglobin levels. Beneficially, the methods reduce or delay exposure to traditional drug therapies (e.g. EPO) and potential side effects associated with such therapies. As a result of reducing or delaying exposure to traditional drug therapies, health care costs, including the cost of therapy, is significantly reduced or at least delayed.

The improved method of treating anemia using Ergothioneine according to the invention may also be administered in combination with traditional drug therapies. In an aspect, the present invention allows for maximized response to EPO and permits use of the lower EPO doses with use of Ergothioneine. The decreased requirements for EPO minimize a patient's risk for thrombosis or thrombotic complications, hypertension, stroke, heart attack and other comorbidities of anemia. Enhanced supplements, compositions and/or pharmaceutical preparations for treating conditions associated with oxidative stress and inflammation, such as anemia, are also disclosed.

According to an embodiment of the invention, a method of treating anemia comprises administering to an animal or patient in need thereof a source of Ergothioneine and a naturally extracted and/or enhanced source of Vitamin D, wherein upon administration of the same improves the treatment of anemia. According to an embodiment of the invention, the enhanced source of Vitamin D may be obtained from a filamentous fungi, tissue, substrate, spent substrate or component thereof, with increased levels of Vitamin D. A suitable example is the novel mushroom whole food Ergo-D2™

According to one embodiment of the methods of treating anemia, the patient in need of treatment thereof has chronic kidney disease (CKD) and/or undergoes hemodialysis (HD). According to these embodiments, the anemia is primarily related to inadequate production of erythropoietin (EPO) with the degree of anemia proportional to the degree of kidney dysfunction. In patients on dialysis, EPO levels are usually lower than in patients with normal kidney function and a similar degree of anemia. The degree of anemia caused by production of less EPO by a diseased kidney in a patient with CKD can be magnified by inadequate bone marrow response due to inflammatory processes in the body. Supporting factors for inflammation is the presence of elevated levels of C-reactive protein (CRP) in pre-dialysis patients. High serum CRP is predictive of a constant inflammatory state during a patient's dialysis program. There is also a prevalence of Vitamin D deficiency in CKD and HD patients, which impairs erythropoiesis due to inflammation. As a result, an embodiment of the invention includes supplementation and restoration of Vitamin D levels in order to stimulate erythropoiesis along with the use of ETT present in red and white blood cell membranes, including mitochondria, so that L-Ergothioneine (ET) can protect erythrocytes against damage related to HbFeIV O (Grundemann, D. H., PNAS, 5256-5261 (2005); Grigat, S. H., Biochem. Pharm., 309-316 (2007)).

Cells that are dividing rapidly are said to be proliferating. Differentiation results in the specialization of cells for specific functions, such as the production of red blood cells by reticulocytes. In general, differentiation of cells leads to a decrease in proliferation. While cellular proliferation is essential for growth and wound healing, uncontrolled proliferation of cells with certain mutations may lead to diseases like cancer. The active form of vitamin D, 1,25-dihydroxyvitamin D, inhibits proliferation and stimulates the differentiation of cells. (Holick M F, Am J Clin Nutr. 79(3):362-371 (2004)).

According to a further embodiment of the invention, the supplementation of Ergo-D2™ to anemia patients, including dialysis patients, provides prophylactic anemia benefits and further results in cost savings and improved health outcome per patient.

Methods of use according to the invention may include administration of the compositions, food products, supplements and/or pharmaceutical compositions on a daily basis, weekly basis, or other frequency for the particular purpose. Although not intending to be limited to a particular theory of the invention, it is believed that daily administration of the Ergothioneine and Vitamin D sources benefit a variety of disease states associated with inflammation and oxidative stress, including anemia. Daily supplementation is preferred for those with significant risk for a particular disease states associated with inflammation and oxidative stress and/or anemia, so that they are preloaded with the bionutrients and have elevated serum levels in order to protect against acute and chronic effects of the conditions. Supplementation on a regular and/or daily basis can also build up storage levels of the key bionutrients which can be mobilized at a time of physiologic need, such as loss of kidney function, infection, inflammation, need for hemodialysis, etc. According to this embodiment, daily supplementation reduces the signs and symptoms of anemia, prevents the comorbidities of anemia and reduces and/or eliminates the need for traditional therapies.

Methods of Use—Diabetes

The World Health Organization estimates that 171 million people worldwide have diabetes and that 340 million will be diabetic by 2030. Ninety percent of current diabetes patients have Type 2 diabetes. The cause(s) of Type 2 diabetes have been linked to inflammation as a causative factor. Inflammation is defined as a response of body tissues to injury or irritation; characterized by pain and swelling and redness and heat. Immune cells in the body, such as macrophages, produce inflammatory molecules, such as cytokines, that can cause inflammation in organs, such as the heart, liver, and islets of Langerhans within the pancreas, while also increasing insulin resistance in muscle, fat tissue and liver. As messengers, cytokines tell other immune cells to activate, grow or even die. Cytokines have the ability to regulate the body's immune system responses and can drive the inflammatory process. There are hundreds of cytokines and their activities can vary, thereby producing different physiologic responses. In the case of the macrophage response, the particular cytokines released cause cells to become insulin resistant, which in turn can lead to Type 2 diabetes. Release of cytokines is part of the inflammatory pathway. Research indicates that disabling the macrophage inflammatory pathway can assist to prevent Type 2 diabetes.

There is also a significant increase in the incidence of Type 1 diabetes, which is considered an autoimmune disease. Type 1 diabetes, formerly known as juvenile diabetes, is an autoimmune disorder where the body loses the ability to produce insulin. It was recently reported that in the prior 8 years the incidence of Type 1 diabetes has increased in youth at a rate of 23%. (Wall Street Journal, Jun. 10, 2012, available at http://www.foxnews.com/us/2012/06/10/concern-as-spike-in-type-1-diabetes-is-seen-in-us-youth/).

The methods of use disclosed herein may be used for treating diabetes and/or other autoimmune disorders. Scientists have hypothesized that the innate immune response of pre-diabetic individuals creates an internal inflammatory response in fat tissue, liver and muscle which leads to insulin resistance and diabetes. In addition, insulin resistance is linked closely to inflammation, namely the pathogenesis of type 2 diabetes. (Shoelson et al., J. Clin. Invest. 116(7):1793-1801 (2006)). In addition, there may be additional or alternative macrophages in people who develop insulin resistance. Macrophages in the adipose tissue, liver, and muscle, as part of innate immunity, secrete pro-inflammatory mediators, creating an inflammation/insulin resistance axis.

The methods of treating diabetes according to the invention beneficially demonstrate modification of the immune macrophage inflammatory response in the liver through the treatment with compositions of the invention, such as a medical food. In an aspect, a medical food composition, such as ErgoD2™, according to the invention preserves essential innate immune functions while at the same time decreasing insulin resistance. Treatment according to the invention provides natural bionutrients, including ergothioneine and Vitamin D2, have the ability to inactivate these inflammatory signaling molecules (i.e. cytokines or free radicals) which are a major contributing factor in insulin resistance. In an aspect of the invention, the methods of treating diabetes result in improved insulin sensitivity. In addition, there is an increase in adiponectin levels which are responsible for regulating glucose metabolism and fatty acid catabolism. Still further, the treatment of diabetes according to the invention may also provider beneficial reductions in Hemoglobin A1C levels as well as increase production of pancreatic insulin.

Methods of use according to the invention may include administration of the compositions, food products, supplements and/or pharmaceutical compositions on a daily basis, weekly basis, or other frequency for the particular purpose. Although not intending to be limited to a particular theory of the invention, it is believed that daily administration of the Ergothioneine and Vitamin D sources benefit a variety of disease states including diabetes. Daily supplementation, including multiple doses per day is preferred, so that a patient is preloaded with the bionutrients and maintains elevated serum levels in order to protect against acute and chronic effects of the conditions. Supplementation on a regular and/or daily basis can also build up storage levels of the key bionutrients which can be mobilized at a time of physiologic need.

According to the invention, daily supplementation reduces the signs and symptoms of diabetes, prevents the comorbidities of diabetes and reduces and/or eliminates the need for traditional therapies. In a preferred aspect of the invention, administering the compositions of the invention control and/or ameliorate symptoms and lower the dosage of oral diabetic drugs.

Methods of Use—Inflammation

The methods of use disclosed herein may be used for various inflammatory diseases and/or conditions associated therewith. According to a further embodiment of the invention, a method of decreasing neuroinflammation and increasing resistance to oxidative stress and associated disease states comprises administering an effective amount of Ergothioneine and a naturally extracted and/or enhanced source of Vitamin D, such as filamentous fungi that has been naturally enriched in Vitamin D2.

A still further embodiment of the invention includes a method of treating a disease state associated with inflammation and/or oxidative stress, including increased production of free radicals comprising administering a composition comprising Ergothioneine and a pulsed UV irradiated, filamentous fungi, tissue, substrate, spent substrate or component thereof, with increased levels of Vitamin D2, wherein upon administration of the same, survivability is increased when compared to an animal with such disease state without such treatment. According to each of the embodiments of the invention the Ergothioneine may be obtained from the whole food sources and/or algae, such as cyanobacteria and Spirulina, as disclosed in this specification.

Demonstrated Efficacy

Applicants demonstrated the use of Ergo-D2™, a potent anti-oxidant, anti-inflammatory nutritional product, to increase numbers and quality of red blood cells and mean corpuscular hemoglobin concentration (MCHC) and decrease total reliance on recombinant erythropoietin. The results provide a natural, cost effective method to control anemia in patients.

Applicants demonstrated that the combination of antioxidants, including phytonutrient turmeric and Ergothioneine, along with Vitamin D enriched mushrooms increase longevity in Drosophila kept under nutritionally deficient diet. These results represent a novel use of the compositions of the invention for treating a variety of disease states associated with inflammation and oxidative stress. According to the invention, Applicants have shown that the compositions increase survival and decrease biologic death in conditions associated with oxidative stress, which include disease states such as Alzheimer's disease and other associated diseases including those involving chronic markers of inflammation, such as chronic depression, traumatic brain injury and PTSD. Thus the supplements, food compositions and pharmaceutical compositions according to the invention, employing the Vitamin D enriched mushrooms, turmeric and Ergothioneine have surprising benefits for treatment of such disease states.

The various embodiments of the invention, including methods of use or administration of compositions for the treatment of inflammation and oxidative stress or disease states or conditions associated therewith, are useful for a variety of subjects. Mammals may be treated using the methods of the present invention and are typically human subjects. According to additional embodiments, the methods of the present invention may be useful for veterinary purposes with other animal subjects, particularly mammalian subjects including, but not limited to, horses, cows, dogs, rabbits, fowl, sheep, and the like. According to additional embodiments, an animal is any non-human primate, such as for example, a cow, horse, pig, sheep, goat, dog, cat, rodent, fish, shrimp, chicken, and the like.

Methods Involving ETT

As confirmed by research into the significance of the ETT, the presence of the transporter (ETT) indicates the presence and/or need for Ergothioneine (ET). (Gründemann, Preventative Medicine, Vol. 54, Supplement 571-574 (May 2012)). This reference is incorporated herein by reference in its entirety. Cells lacking ETT are unable to accumulate ET, as a result of the plasma membrane being virtually impermeable for the hydrophilic zwitterion compound of ET. As a result, the existence of the ETT indicates the clear beneficial role for ET as set forth according to the various embodiments of the invention. Immunohistochemistry studies set forth in the Examples of the invention demonstrate that certain cells have strong expression of ETT. According to the methods of the invention, the cells with strong expression of ETT are capable of accumulating ET to higher levels. For conditions disclosed herein, including anemia and diabetes, the accumulation of ET may be critical to treating these disease states and the associated conditions.

According to an embodiment of the invention, the ability to detect the presence, absence, and/or concentration of ETT can be a diagnostic and/or therapeutic method according to the various embodiments of the invention. The diagnostic identification and measurement of the ETT within the membranes of specific cells and/or mitochondria related to various diseases and conditions. Additional description of diagnostic methods is provided in U.S. Application Ser. No. 61/628,162 entitled “Application of the Ergothioneine Transporter SLC22A4 and/or L-Ergothioneine to Targeted Diagnostic Identification and Treatment of Autoimmune Diseases,” which is herein incorporated by reference.

In an aspect of the invention, the absence, presence or specific concentration of ETT, the protein transporter encoded by SLC22A4, in cells may be significant in terms of susceptibility to a particular disease and/or potential to regulate such disease. Polymorphisms of SLC22A4 have been implicated in disease states associated with specific populations, such as rheumatoid arthritis in the Japanese population and with Crohn's disease in a Canadian cohort. (Newman, B. et al., Arthritis & Rheumatism, Volume 52, Issue 2, pages 425-429, February 2005). It is an object of the present invention that dosing to particular individuals of ET as part of personalized medicine can lead to modulatory changes in translation of messages from genetic DNA with resultant repair of a disease process.

In a further aspect of the invention, the amino acid L-Ergothioneine, with and without the help of Vitamin D2 has the ability to control and/or modify the transcriptional process. As one skilled in the art shall ascertain, transcription and translation are the steps through which a functional protein is synthesized from the genetic material DNA. These processes are found to occur both in prokaryotes (organisms that lack a cell nucleus or other membrane bound cell organelles) or as well as eukaryotes (organisms that have a cell nucleus). Transcription is the first stage of the expression of genes into proteins. In transcription, an mRNA (messenger RNA) intermediate is transcribed from one of the strands of the DNA molecule. The RNA is called messenger RNA because it carries the ‘message’ or genetic information from the DNA to the ribosomes, where the information is used to make proteins. Translation is the process which follows the transcription event. The primary transcript is translated into a sequence of corresponding amino acids forming a peptide chain. These undergo further processing and folding to form the final fully functional proteins. Translation is the process of making peptide strands from primary transcript. There are a set of amino acids which are carried to the site of translation by specific transfer RNAs for the process. Apart from this messenger RNAs and ribosomal RNAs also play significant roles in translation.

The processes of transcription and translation further differ in their regulation. Transcription is highly regulated by internal mechanisms based on chromatin structure, histones, DNA methylation etc. in eukaryotes and operon mechanisms. The operon regulation involves promoter sequences/activators and suppressors which are found in the sequence. Alternatively, translational control is mainly through regulation of binding of ribosomal subunits to the translation complex. Most naturally occurring antibiotics, toxins and drugs target this process. In addition, the post event modifications differ between the processes. Transcriptional product undergoes splicing and dicing events that remove the intragenic portions (introns) which are non-coding in nature. Alternatively, post translational modifications are mainly chemical in nature attaching functional groups to the peptide sequence.

The enzymes involved in transcription and translation further differ as well as the location of the events. A single RNA polymerase is found to be capable of carrying out and controlling the transcription in prokaryotes and three such enzymes are at work in eukaryotes. Alternatively, translation requires several enzymes and factors for the process. It has mainly three steps, initiation, elongation and termination each of which requires a set of RNAs, cofactors and enzymes. Site transcription generally occurs in the nucleus where the transcription factors and enzymes are available. Translation on the other hand occurs in the cytoplasm after the primary mRNA transcript is transferred from the nucleus to the cytoplasm.

The events transcription and translation can be considered as two consecutive processes in production of a functional protein. Both events are controlled by different factors and enzymes but eventually work toward the same goal. Though the regulation, mechanism and other factors differ both are targets for drug designing since they are being controlled by rigorous mechanisms.

It is an embodiment of the present invention, that the amino acid L-Ergothioneine, with and without the help of Vitamin D2 has the ability to control and/or modify the transcriptional process.

Paul and Snyder state: “ET protects cellular DNA from damage induced by reactive oxygen species. ETT is abundantly expressed in mitochondria. Mitochondrial DNA is especially vulnerable to stress, because unlike nuclear DNA, there are no histones to protect it. Mitochondria also lack the very efficient DNA repair mechanisms of the nucleus. The electron transport chain of mitochondria generates free radicals and ROS, such as superoxide and hydroxyl radical that create redox imbalance. In the process, mitochondrial DNA itself is targeted by ROS leading to DNA nicks, breaks and mutations. A region of the mtDNA, the Displacement or D-loop, is a hotspot for DNA damage. Several mutations occurring here are associated with cancers.” (Paul & Snyder, Cell Death and Differentiation (2009), 1-7, Macmillan Publishers Limited).

These scientists further conclude their study provides substantial evidence that ET is a physiologic antioxidant cytoprotectant. ET tissue levels are maintained by its transporter, ETT. Depletion of ETT by RNA interference prevents the antioxidant actions of exogenous ET. More importantly, in the absence of added ET, ETT depletion leads to enhanced oxidative damage of protein, lipid and DNA as well as augmented cell death. In these studies the incubation media contained very low concentrations of ET so that cytoprotection was afforded by ‘endogenous’ ET accumulated by the cells.

Such evidence indicates that ET is a most unusual amino acid with substantial antioxidant efficacy. The existence of a physiologic ET transporter is responsible for high tissue levels. Depletion of ETT leads to augmented oxidative stress and cell death. ET preferentially protects water-soluble proteins from oxidative damage. The high density of ETT in mitochondria implies a unique role in protecting this organelle from the reactive oxygen species that accumulate even with normal oxidative metabolism. ET also protects the cell from damage induced by reactive nitrogen species and UV radiation. For all these reasons ET appears to be an important physiologic cytoprotectant which probably merits designation as a vitamin.

The repair of diseased tissues leads to rapid cell division and differentiation of reactive stem cells. This repair process involves production of toxic byproducts of cellular metabolism, such as cytokines (free radicals). As set forth in the description of the invention, various scientists have promoted the importance of ET in detoxification of these toxic free radicals. It is a further aspect of the invention that ETT is increased in concentration in reactive repair cells to bring needed amounts of ET for neutralization of these toxic free radicals that cause cell death, as described further in Example 4 (study showing control of Paraquat induced oxidative stress/biologic death by ErgoD2™). In a further aspect of the invention, dosing of the medical food ErgoD2 or foods containing Ergothioneine and enhanced levels of vitamin D2 have the potential to increase the production of ETT within the cellular membranes of reactive stem cells as part of the disease repair process, especially in autoimmune diseases, such as diabetes, rheumatoid arthritis and in certain conditions associated with anemia. As recognized according to the invention, ET tissue levels are maintained by ETT—the transporter, and depletion of ETT by RNA interference prevents the antioxidant actions of exogenous ET.

In a still further aspect of the invention, methods for correcting or modifying genetic polymorphism of the gene with ET dosing may be employed. ETT is highly concentrated in the plasma membrane and mitochondria. Until the recent immunohistochemistry studies performed by us with antibodies for ETT the detailed intracellular localization of ETT was not available for analysis and study. Several facts underscore the role of ET and ETT in DNA protection. The importance of ET is shown by the fact that movement of ET by ETT into cells is a one way mechanism. Once inside, the cell holds on to this important amino acid molecule. In addition, as recognized by Paul & Snyder: “in the absence of added ET, ETT depletion leads to enhanced oxidative damage of protein, lipid and DNA as well as augmented cell death.” As a result, cytotoxicity and DNA polymorphic changes can be caused by the generation of toxic free radicals such as copper 2⁺, iron 3⁺, cytokines, etc. The neutralization of these toxic free radicals by ET electron donation can lead to correction of DNA polymorphism through modification of the translation process as previously described.

All publications and patent applications in this specification are indicative of the level of ordinary skill in the art to which this invention pertains. All publications and patent applications are herein incorporated by reference to the same extent as if each individual publication or patent application was specifically and individually indicated by reference.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

Experiments testing the anti-inflammatory effects of mushroom-based formulations with increased natural levels of Ergothioneine according to the invention were tested in an equine inflammatory gum disease study. Elderly horses with inflammatory gum were treated with a mushroom-based formulation, 10 grams per day, for 30-60 days; formulations used were selected because of increased natural levels of Ergothioneine. Horses showed dramatic improvement in the severity of the gum disease within 30-60 days as shown in FIG. 1.

### Example 2

White Blood Cell (WBC) Study

A separate 30 day clinical animal study, involving 36 horses, fed mushroom-based formulations, revealed a statistically significant increase in numbers of white blood cells; mean response among the study sample was 12%. The results are shown in FIG. 2. This percentage increase in white blood cells within a 30 day period after dietary supplementation is further supportive evidence for improvement in the animal's immune response and ability to suppress inflammatory diseases, such as gum disease.

Inflammatory disease of the gums is a perfect example of the inflammatory process that occurs in other tissues and/or organ systems, such as arteries, nerves, heart, colon, and brain, to name a few. The terms inflammation, free radicals, reactive oxygen species (ROS) and oxidative stress are almost interchangeable and a clear understanding of the interactive processes has uncovered new approaches to prevention and amelioration of inflammation and or inflammatory disorders no matter what the origin or location. Similar to the inflammatory processes involved in gum disease, free radicals can perpetuate tissue and organ damage and the disease itself.

The primary function of ET is the protection of RBCs against damage related to ferryl hemoglobin. Monocytes do not express hemoglobin and the roll of ET may be another target, such as peroxidases. (Grigat, S. H., Biochem. Pharm., 309-316 (2007); Lagorce J F, Pharmacology, 173-178 (1997)). ET appears to provide protection for monocytes by specific interaction with peroxidase(s). The lack of ET may represent a precipitating factor in the genesis of chronic inflammatory disease (Gründemann, PNAS, 5256-5261 (2005)).

Red Blood Cell (RBC) Study

In the same pilot animal study described above (White Blood Cell Study), the horses also showed a 7.6% mean corpuscular hemoglobin concentration (MCHC) increase within 30 days as shown in FIG. 3. RBCs have a 120 day lifespan and we were quite excited by this quick increase in MCHC. The role of free radicals and heme degradation supports the results (Alayash, A., Nat. Rev., 152-159) (2004)).

ET is distinguished from other antioxidants in its interaction with protein-bound heme. No affects are expected on native hemoglobin (HbFeII) by ET, rather ET binds to or react with ferryl hemoglobin (HbFeIV O). The HbFeIV O species is a highly reactive intermediate in the autocatalytic oxidation, caused by many xenobiotics, of HbFeIIO2 to methemoglobin (HbFeIII) and is also considered a starting point for detrimental radical reactions including heme degradation. As supported by the work of Gründemann, the primary function of ET is protecting erythrocytes against damage related to HbFeIV 0, demonstrating a role in anemia treatments and prevention.

In addition, the study confirmed the LD50 is at least 50-100 times the recommended human dose for the compositions according to the invention. As a result, the tested products are considered non-toxic. Anecdotal reports from people regarding improvements in levels of RBCs, WBCs, and MCHC have filtered in with use of other mushroom-based products, such as ImmuSANO™ and GlucoSANO™ according to additional embodiments of the invention using the compositions described herein.

### Example 3

Pulses of UV radiation of approximately 1-10 J/cm² per pulse, preferably 3-8 J/cm² and most preferably 5-6 J/cm² are used to UV-enhance Vitamin D and/or its derivatives in filamentous fungi. Voltages may also vary based upon safety concerns but should generally be in the range of 1 to 10 or even up to 100 or 10,000 volts as safety mandates. The pulses should generally be in a range of 1-50 pulses per second more preferably 1-30 pulses per second and most preferably 1-10 pulses per second for a range of treatment post-harvest of 0 to 60 seconds.

The inventors used 5.61 J/cm² per pulse on the strobe surface for an input voltage of 3800V and with 3 pulses per second. Sliced mushrooms (Agaricus bisporus, white strain) were placed in the pulsed UV-light sterilization chamber and treated with pulsed light for up to a 20-second treatment at a distance of 17 cm from the UV lamp or 11.2 cm from the window. Control samples did not undergo any pulsed UV treatment. Treated mushrooms were freeze-dried and then sent to a selected commercial laboratory for Vitamin D2 analysis. In this study, a pulsed UV system was also evaluated for effects on the appearance of fresh mushroom slices during a shelf life study.

Results of the experiments demonstrated that pulsed UV-light was very effective in rapidly converting ergosterol to Vitamin D2. Control mushrooms contained 2 ppm d.w. Vitamin D2, while 10 and 20 seconds of exposure to pulsed UV-light resulted in 17 and 26 ppm Vitamin D2, respectively (FIG. 3). This increase was equivalent to over 1800% DV Vitamin D in one serving of fresh mushrooms after a 20 second exposure to pulsed UV (FIG. 5). The mushrooms treated for 20 seconds also showed no noticeable difference in appearance initially as well as after 10 days of storage at 3° C. compared to the untreated control.

These results compared favorably to the previous pilot study (Feeney, 2006) where mushrooms were exposed to 5 minutes of conventional UV-light exposure. In that study, the mushrooms contained 14 ppm Vitamin D2, but they were also significantly discolored. Hence, the pulsed UV method shows considerable promise as a rapid means to enhance Vitamin D2 levels in fresh mushrooms, theoretically reducing required exposure times from minutes to seconds. Pulsed UV-light exposure did not result in any negative effects on mushroom quality.

Another experiment revealed that pulsed UV-light could rapidly convert ergosterol present in dried oyster mushroom powder to Vitamin D2 (Table 1). These findings indicate that this technology could be used to enrich other mushroom products with Vitamin D2.

TABLE 1
Vitamin D2 generation in dried oyster mushroom powder
exposed to pulsed UV-light (C-type lamp).

| Time of Exposure(s) | Vitamin D2 (PPM) |
|---|---|
| 0 | 8.5 |
| 8 | 15.18 |
| 16 | 24.24 |

The filamentous fungi product is subjected to pulsed UV irradiation after harvest, being irradiated with UV light for a time sufficient to enhance the Vitamin D content thereof. By utilizing UV irradiation, the food product has a substantially increased level of Vitamin D. Preferably, the food product is irradiated with UV radiation, specifically Ultraviolet-B (UV-B), a section of the UV spectrum, with wavelengths between about 280 and 320 nm, or Ultraviolet-C (UV-C), with wavelengths between about 200 and 280 nm. In a more preferred embodiment the UV radiation is pulsed. It is believed that the additional Vitamin D is obtained through the conversion of ergosterol due to the UV irradiation. The time may be the same or increased when the irradiation occurs during the growing process, or post-harvest though the UV irradiation can occur during both periods.

### Example 4

The effect of Agaricus blazei (1-4) on the survival rate of Drosophila melanogaster fed a nutritionally deficient diet, at room temperature (22° C.) was tested using the following parameters: Agaricus blazei (no UV treatment): 1.6 g Vitamin D2/g, dry weight; two pulses of UV-B light: 241.0 g Vitamin D2/g, dry weight; plain yeast paste base as control; vials containing 5.0 ml 1% Agarose medium; yeast paste containing 3% w/w concentration of the two samples.

Drosophila is a model organism with an experimental history of over 100 years. It has a life cycle (embryo to adult) of about 12 days at 22° C. and 9 days at 25° C. The adults live for about 85 days at 22° C. and 60 days at 25° C. under laboratory conditions. It has 3 major chromosomes. Drosophila and human development are homologous processes. They utilize closely related genes working in conserved regulatory networks. Unlike humans, Drosophila can be genetically manipulated. As a result, most of what we know about the molecular basis of animal development has come from studies of model systems such as Drosophila. Drosophila has nearly all the important genes that vertebrates including humans have. Not only the genes are conserved but the pathways regulated by these genes are also conserved. A reliable model using Drosophila as a system to evaluate the effect of a compound for survival on nutritionally deficient diet has been developed by Dr. Krishna Bhat.

The effect of Agaricus Blazei without enrichment, with Vitamin D2 enrichment, pure Vitamin D2 and control (vehicle for the delivery) on the survival rate of Drosophila melanogaster under Paraquat-induced oxidative stress condition was tested. The study focused on the control of Paraquat induced oxidative stress/biologic death. Paraquat is a very potent oxidative stress inducing chemical and causes death in animals and plants by the toxicity of released free radicals. Paraquat (10 mM concentration) (Sigma Aldrich) was used to chemically induce oxidative stress. Paraquat is the trade name for N,N′-dimethyl-4,4′-bipyridinium dichloride, a widely used herbicide. Paraquat, a viologen, is quick-acting and non-selective, killing green plant tissue on contact. It is also toxic to humans when swallowed. This is the most standard chemical used in experimental induction of oxidative stress using the Drosophila model system. It catalyzes the formation of reactive oxygen species (ROS). Paraquat will undergo redox cycling in vivo, gets reduced by an electron donor such NADPH, before being oxidized by an electron receptor such as dioxygen to produce superoxide, a major ROS.

The following materials and methods were utilized. Vials containing 10 mM Paraquat (from Sigma Aldrich) in 5 ml of 1.2% Low melting point Agarose medium were prepared. A strip of half moist filter paper was inserted in the medium (with the wet end in). Yeast paste containing 1% concentration (w/w) of the various test materials (see above) mixed to homogeneity was prepared. Yeast paste without drug was used as control. Uniform aliquot (˜300 mg) of yeast paste with or without the test material) was applied to vials in such a way that yeast paste was on the glass surface and covered the dry end (top) of the filter paper strip. Freshly enclosed wild type isogenized Canton-S males and females were collected and starved on 1% agar medium for 5-6 hours. Four males and females were transferred to the vial containing 10 mM Paraquat in LMP agarose medium and yeast paste with +/− test material (8 flies per vial). 6 vials were used per experiment. Vials with flies were placed horizontally in a tray. The experiment was conducted at 25 degrees C. temperature. Flies were transferred once in 2 days and the number of flies surviving at each transfer was recorded.

Results: Over a period of 10 days, flies fed yeast paste containing A. blazei with Vitamin D2 enrichment showed marked and significant survivability under Paraquat-induced oxidative stress condition compared to the control yeast paste alone (54%+/−10% versus 23%+/−8%), yeast paste containing A. blazei without the Vitamin D2 enrichment (54%+/−10% versus 27%+/−8%), and yeast paste containing pure Vitamin D2 (54%+/−10% versus 13%+/−3%). Vitamin D2 in its pure form had a deleterious effect on the survival and therefore seems to aggravate the oxidative stress.

The results show that a combination of naturally induced Vitamin D2 together with the components of A. blazei has the highest potential and activity to suppress the oxidative stress from Paraquat. These results are highly significant; showing that Vitamin D2, produced naturally by mushrooms, was active only when present within the parent whole food; Vitamin D2 and Vitamin D3 by themselves (i.e. single nutrient or pure Vitamin D2 and Vitamin D3) had no beneficial effect. Oxidative or inflammatory stress was dramatically induced in the Drosophila fruit fly model by the toxic agent, Paraquat, and the end-point of death was evaluated. This model is a very well established paradigm to evaluate oxidative stress. These findings show a novel use for A. blazei enriched with Vitamin D2 for suppressing oxidative stress and associated biologic death. The results are shown in FIGS. 4 and 5.

### Example 5

A. blazei enriched with Vitamin D2 significantly were analyzed to determine whether they enhance the survival and life span of Alzheimer's disease (AD) model in Drosophila. The study evaluated the ability of the edible specialty mushrooms according to the invention, with and without naturally enhanced levels of organic Vitamin D2, to extend the lifespan of the Alzheimer's disease mutant fruit fly.

Type of Model (with specific Drosophila model of neurodegeneration). The targeted over/ectopic expression of APP in the brain using a UAS promoter driven APP transgene, induced by a specific GAL4 trans-driver in the brain of a Drosophila model system, was used for this Example. Below is a reference for such over-expression of APP in the Drosophila model system and the combination gives a fully penetrant AD with limited life-span.

β-Amyloid peptides and amyloid precursor protein (APP) play a deterministic role in Alzheimer's disease (AD). In Drosophila, the targeted expression of the key genes of AD, APP, causes generation of β-amyloid plaques and age-dependent neurodegeneration as well as progression to semi lethality, a shortened life span; genetic manipulations or pharmacological treatments with secretase inhibitors influenced the activity of the APP-processing proteases and modulated the severity of the phenotypes (Greeve I., et al., J. Neuroscience 24, 3899-3906 (2004)). The AD strain lives only for a few days after their eclosion (birth) as opposed to 65 days or more for wild type normal strains.

We determined the extension of life span in the mutant strain for each test compound. We used a specific GAL4 driver that induces the APP gene in the central brain area at high levels (see above) and results in a fully penetrant lethality within a 2-3 weeks period. When these AD flies are given A. blazei enriched with Vitamin D2, the survival rate was increased nearly double that of the control or A. blazei without any enrichment. See FIG. 5. Treating AD flies with pure Vitamin D2 or Vitamin D3 had no such effect. These results indicate that components in A. blazei, in combination with UV-enriched natural Vitamin D2, have significant benefit against the AD disease.

The results show the ability of a proprietary whole food mushroom with naturally enhanced vitamin D2 to dramatically decrease death by 27%. Statistically significant findings also revealed a physiologic difference between activity of synthetic forms of Vitamin D2 and Vitamin D3 in this neurodegenerative disease model. Study results suggest that neurons may have both Vitamin D2 and Vitamin D3 receptors and that these neuronal cell receptors may be more responsive to Vitamin D2 as compared to Vitamin D3.

### Example 6

The work of Gründemann et al. demonstrates additional sources of ET biosynthesis, including species of cyanobacteria (synthesis confirmed by the detection of the intermediate hercynine). The highest ET content of cyanobacteria in the examined samples was close to 1 mg per g dry mass, which approaches the same level as the top values (1-2 mg per g dry mass) reported previously for several mushrooms. As a result, it is demonstrated that cyanobacteria are a “high density” source of ET.

Previously, the biosynthesis of ET has been demonstrated only in fungi (including edible mushrooms) and mycobacteria, but these are unlikely sources for fishes. In the present study, the origin of ET accumulated in zebra fish was examined. There was virtually no ET, measured by LC-MS, in most tank vegetation (plant, green and red alga). However, ET was detected in a Phormidium sample, a cyanobacterium. In commercial fish feed preparations, ET content increased with the content of cyanobacteria Arthrospira platensis or Arthrospira maxima (Spirulina). High levels of ET (up to 0.8 mg per g dry mass) were measured in cyanobacteria preparations sold as dietary supplements for humans and in fresh Scytonema and Oscillatoria cultures. Thus, cyanobacteria can contain as much as or even more ET than King Oyster mushrooms (Pleurotus eryngii) which we measured at 0.4 mg per g dry mass. All samples with substantial ET content also contained the biosynthesis intermediate hercynine; this strongly suggests that cyanobacteria synthesize ET de novo and can produce high levels of Ergothioneine. Spirulina is a novel, safe, accessible, and affordable source of Ergothioneine for humans.

### Example 7

Human studies are conducted to evaluate the use of medical foods for treating diabetes and anemia. An exploratory study is underway to further evaluate the clinical tolerability and potential therapeutic benefits of treating patients with diabetes and anemia with ErgoD2™ Hemo, a 2000 mg ErgoD2 medical food composition (ergocalciferol (Vitamin D2 11,000 IUs and L-ergothioneine 3 mg). The treatment of patients with ErgoD2™ Hemo provides a composition having naturally high concentrations of the powerful antioxidants L-Ergothioneine and Ergocalciferol (vitamin D2), which work together to naturally elevate red blood cell production and decrease insulin resistance, enabling the body to more easily respond to symptoms experienced by the vast majority of patients taking prescription drugs to treat these conditions.

The human studies are measuring how ErgoD2™ Hemo affects each patient's normal standard of care over 90-120 days. Clinical response biomarkers will include Vitamin D2 and D3 levels, hemoglobin A1C (stable marker of diabetes severity), changes in diabetic drug dosing, dosing of red blood cell substitutes, such as Epogen™ (manufactured by Amgen) in dialysis patients, and patient symptomatic response. The measurement of the impact on use/dosage of injectable and/or oral drugs for diabetes and anemia represents a significant clinical benefit of the methods of the invention. The clinical response biomarkers will include lab measurements at baseline and on days 0, 30, 60, 90, etc. for all blood data, which includes CBC with MCHC, glucose, hemoglobin A1C, and insulin, cholesterol and lipoproteins, iron saturation, ferritin, calcium, phosphorus, albumin, and D-25 levels (D2 and D3).

### Example 8

The use of ergothioneine and the compositions of the invention for treatment of anemia, including anemia caused by kidney disease, is supported by scientific evidence that the hormone erythropoietin (EPO) is produced by the kidney, mainly in proximal convoluted tubular cells. When it is produced by the kidney, it travels to the bone marrow and initiates maturation of red blood cells. As a result, without EPO the production of red blood cells is diminished.

Immunohistochemistry studies according to the invention demonstrate the role of ETT and Ergothioneine in bone marrow and the kidney. Antibody titration experiments were conducted with a proprietary rabbit polyclonal antibody to SLC22A4 using steam-based antigen retrieval (pH 6.0 citrate buffer) to establish concentrations that would result in minimal background and maximal detection of signal. Serial dilutions were performed at 20 ug/ml, 10 ug/ml, 5 ug/ml, and 2.5 ug/ml using the antibody on formalin-fixed, paraffin-embedded human tissues supplied by LifeSpan Biosciences and control cell lines (ETTh and CTTh) supplied by Entia Biosciences, Inc. (Dr. Dirk Gründemann) prepared by LifeSpan. The principal detection system consisted of a Vector anti-rabbit secondary (BA-1000) and a Vector ABC-AP kit (AK-5000) with a Vector Red substrate kit (SK-5100), which was used to produce a fuchsia-colored deposit. Tissues were also stained with positive control antibodies (CD31/vimentin cocktail) to ensure that tissue antigens were preserved and accessible for immunohistochemical analysis. Only tissues that were positive for CD31 and vimentin staining were selected for the remainder of the study. The negative control consisted of performing the entire immunohistochemistry procedure on adjacent sections in the absence of primary antibody. The slides were interpreted by a pathologist and each antibody was evaluated for the presence of specific signal and level of background. Staining was recorded on a 0-4 scale (0=negative, 1=blush, 2=faint, 3=moderate, 4=strong). Slides stained at 2.0 ug/ml were imaged with a DVC 1310C digital camera coupled to a Nikon microscope. Images were stored as TIFF files with Adobe Photoshop.

Using the antibody described in these methods immunohistochemistry results shows the color red/fuchsia indicating the presence of the SLC22A4 gene (i.e. the Ergothioneine Transporter (ETT), which is a peptide). The red-fuchsia staining shown in FIG. 7 indicates the strong expression of ETT in normal bone marrow of reticulocytes which are the precursor cells for red blood cells. In FIG. 8 kidney tissue, namely the proximal convoluted cells (PCT) also show strong staining indicating the presence of ETT. The strong expression in the PCT cells in the kidney demonstrates the cells responsible for producing the hormone EPO.

This activity of the ETT and ergothioneine in erythrocyte progenitor cells (FIG. 7) and tubular epithelial cells (FIG. 8) demonstrate that ergothioneine is necessary for the increased production of red cells and erythropoietin. As a result the methods of the invention providing ergothioneine and/or compositions of the invention provide beneficial clinical effects for increasing production of red cells and erythropoietin to treat a patient having anemia.

As confirmed by research into the significance of the ETT, the presence of the transporter (ETT) indicates the presence and/or need for Ergothioneine (ET). (Gründemann, Preventative Medicine, Vol. 54, Supplement 571-574 (May 2012)). As a result, the immunohistochemistry data indicating the presence of the ETT indicates the importance of treatment methods according to the invention, namely to provide Ergothioneine for production of EPO and the production of red blood cells. In addition, the presence of rapidly dividing cells results in some toxic byproducts of metabolism being produced and the methods of the invention, providing Ergothioneine help to neutralize toxic free radicals in order to promote cell survival as opposed to normal metabolism leading to general cell death (i.e. apoptosis).

### Example 9

The use of ergothioneine and the compositions of the invention for treatment of diabetes are also supported by scientific evidence that the production of glucagon and insulin in the pancreas is impacted by the presence of the ETT and therefore ergothioneine. Immunohistochemistry studies according to the invention demonstrate the role of ETT and Ergothioneine in the islets of Langerhans (pancreas cells) (Study done at Lifespan Biosciences, Seattle, Wash.). According to the methods of Example 8, using an antibody that specifically stains the Message from SLC22A4, immunohistochemistry shows the color red/fuchsia indicating the presence of the SLC22A4 gene (i.e. the Ergothioneine Transporter (ETT)).

The faint red-fuchsia staining shown in FIG. 9 indicates the faint expression of ETT in normal pancreas tissue, namely the islets of Langerhans responsible for the production of glucagon and insulin. In comparison, FIG. 10 shows the strong expression of ETT in a pancreas of a diabetic patient. This activity of the ETT and ergothioneine in pancreas cells of a diabetic patient demonstrates that ergothioneine is necessary for the production of glucagon and insulin and/or plays a role in the body's mechanism of repair of these damaged tissues. As a result the methods of the invention providing ergothioneine and/or compositions of the invention provide beneficial clinical effects for improving insulin sensitivity and treating diabetes.

### Example 10

The presence of increased concentrations of the ETT is widely apparent in bodily tissues and/or cells involved in the autoimmune process. This includes for example, rheumatoid arthritis, allergic rhinitis, type 1 diabetes mellitus, Psoriasis, and alopecia areata. By comparison, normal tissues and diseased non-autoimmune tissues showed much less presence of the ETT. The potential reparative activity of Ergothioneine in autoimmune diseases, including type 1 and type 2 diabetes, is supported by examples in normal joints and rheumatoid arthritis joints using the methods disclosed in Example 8.

As shown in FIG. 11 and FIG. 12 synoviocytes and subsynovial histiocytes of a normal, healthy joint show negative to faint staining, indicting the lack of ETT in the tissues. The vascular smooth muscle was faintly positive, whereas fibroblasts were negative. In comparison, as shown in FIG. 13 and FIG. 14 synoviocytes and subsynovial histiocytes of a patent having rheumatoid arthritis show moderate to patchy focal strong staining in the tissue, indicting the increased presence of ETT in the tissues. In addition, reactive capillaries were moderately positive; infiltrating macrophages were strongly positive; plasma cells were moderate to strong; lymphocytes were faint. The rheumatoid arthritis sample showed significantly increased staining of reactive synoviocytes and subsynovial histiocytes, and strong staining of infiltrating macrophages, as well as increased staining of reactive fibroblasts and capillaries.

### Example 11

The presence of increased concentrations of the ETT is widely apparent in bodily tissues and/or cells involved in the autoimmune process. This finding further includes Crohn's disease. By comparison, normal tissues and diseased non-autoimmune tissues showed much less presence of the ETT. The potential reparative activity of Ergothioneine in autoimmune diseases, including type 1 and type 2 diabetes, is supported by examples in patient's having Crohn's disease using the methods disclosed in Example 8.

As shown in FIG. 15 and FIG. 16 sections of a normal, healthy small intestine show faint staining. The absorptive epithelium was faintly to moderately positive, and goblet cells showed faint staining Plasma cells within the lamina propria showed moderate staining, and macrophages were moderate. Vessels within the submucosa showed faint to moderate staining of endothelium and faint staining of smooth muscle. Within enteric ganglia, ganglion cells were faint to moderate and Schwann cells were blush to faint. Smooth muscle of the muscularis mucosa and muscularis propria were blush positive, and fibroblasts were faint.

In comparison, as shown in FIG. 17 and FIG. 18 showed small intestine with changes consistent with Crohn's disease. Reparative epithelium and epithelium deeper in crypts showed variable faint to moderate staining Plasma cells in the lamina propria were positive. Collections of histiocytes were moderately positive. In areas of ulceration, collections of macrophages and plasma cells were moderately to strongly positive. Lymphocytes were mostly negative. In areas of inflammation, reactive capillaries showed moderate staining of endothelial cells. Muscular vessels within the submucosa showed faint staining within endothelial cells and vascular smooth muscle. Within enteric ganglia, ganglion cells were faint to moderate and Schwann cells were blush to faint. Compared to normal colon (FIGS. 15 & 16), samples showing inflammation consistent with Crohn's disease showed increased staining of plasma cells and macrophages in areas of inflammation and ulceration, with increased staining of reactive capillaries.

1. A method of treating anemia and/or diabetes in mammals comprising:
administering to said mammal in need of treatment thereof a source of Ergothioneine and Vitamin D; and
neutralizing free radical damage and promoting blood cell development in said mammal.

2. The method of claim 1 wherein said Vitamin D is Vitamin D₂ and/or Vitamin D₃.

3. The method of claim 1 further comprising obtaining a source of Ergothioneine from a whole food and/or bacteria source.

4. The method of claim 3 wherein said source is the cyanobacteria Spirulina or a cereal crop (e.g. oat, barley, hops).

5. The method of claim 1 wherein said source of Ergothioneine and Vitamin D is a naturally enhanced, filamentous fungi, tissue, substrate, spent substrate or component thereof.

6. The method of claim 5 wherein said filmentous fungi is a mushroom of a species selected from the group consisting of: Agaricus bisporus, Agaricus blazei, Lentinula edodes, and Pleurotus ostreatus, and wherein said mushroom is enriched by pulsed UV irradiation without changing said mushroom's Ergothioneine content.

7. The method of claim 5 wherein said fungi is in powder form.

8. The method of claim 1 wherein said administering step further includes providing a source of erythropoietin or erythropoiesis-stimulating agent.

9. The method of claim 2 wherein said Vitamin D₂ content is increased to about 800% of the daily recommended value of Vitamin D.

10. A method of treating a disease state associated with inflammation, oxidative stress and damage to blood cells and associated disease states in mammals comprising:
administering to said mammal an effective amount of Ergothioneine and a filamentous fungi that has been naturally enriched in Vitamin D₂, wherein upon administration of the same, survivability of said animal is increased, progression of the disease state of said animal is decreased and/or need for additional drug therapy is reduced when compared to an animal with such disease state without such treatment.

11. The method of claim 10 wherein said enrichment is from UV treatment and said filamentous fungi is a mushroom selected from the group of species consisting of: Agaricus bisporus, Agaricus blazei, Lentinula edodes, Pleurotus ostreatus and Pleurotus eryngyi and wherein said Vitamin D₂ content is increased to about 800% of the daily recommended value of said vitamin.

12. The method of claim 10 further comprising obtaining a source of Ergothioneine from a whole food source, a cereal crop (e.g. oat, barley) and/or bacteria source (e.g. cyanobacteria Spirulina).

13. The method of claim 10 wherein said Ergothioneine, with or without Vitamin D2 is capable of controlling and/or modifying the transcriptional and/or translational process.

14. The method of claim 10 wherein said methods correct or modify genetic polymorphisms of the ETT gene as a result of providing said Ergothioneine.

15. A nutritional product for treating anemia and/or diabetes and preventing comorbidities of anemia and/or diabetes in animals comprising:
Ergothioneine; and
a source of Vitamin D.

16. The nutritional product of claim 15 further comprising an antioxidant and/or a phytonutrient.

17. The nutritional product of claim 15 wherein said source of Vitamin D is a UV irradiated, filamentous fungi, tissue, substrate or component thereof with higher levels of Vitamin D than a non-irradiated product, and wherein said Ergothioneine is obtained from a whole food and/or bacteria source.

18. The nutritional product of claim 15 wherein said nutritional product increases the production of ETT within cellular membranes of reactive stem cells (e.g. part of the disease repair process), including stem cells in a patient having an autoimmune disease (e.g. diabetes, rheumatoid arthritis and/or conditions associated with anemia).

19. A pharmaceutical composition for treating anemia and/or diabetes and preventing comorbidities of anemia and/or diabetes in animals comprising:
a source of Ergothioneine;
a source of Vitamin D;
a source of erythropoietin or erythropoiesis-stimulating agent;
a pharmaceutically-acceptable carrier; and
optionally an antioxidant and/or phytonutrient.
